(19) United States
(12) Patent Application Publication
(10) Pub. No.: US 2010/0247707 A1
(43) Pub. Date: Sep. 30, 2010

(54) DIPEPTIDES AS FEED ADDITIVES

(75) Inventors: Christoph KOBLER, Alzenau (DE); Thomas HAEUSSNER, Bad Orb (DE); Katja KELM, Niedernberg (DE); Christoph WECKBECKER, Gruendau-Lieblos (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: EVONIK DEGUSSA GmbH, Essen (DE)

(21) Appl. No.: 12/750,103

(22) Filed: Mar. 30, 2010

Related U.S. Application Data
(60) Provisional application No. 61/238,316, filed on Aug. 31, 2009.

(30) Foreign Application Priority Data
Mar. 31, 2009 (DE) 102009002044.6

(51) Int. Cl. A23K 1/16; A23K 1/18; C07C 271/22; C07D 233/64; C07D 209/20; C07C 321/02; C07K 1/00

(52) U.S. Cl. 426/2; 426/656; 560/148; 560/16; 548/338.5; 548/495; 562/556

(57) ABSTRACT

The invention relates to feed additives containing dipeptides or salts thereof, in which one amino acid residue of the dipeptide is a DL-methionyl residue and the other amino acid residue of the dipeptide is an amino acid in the L-configuration selected from lysine, threonine, tryptophan, histidine, valine, leucine, isoleucine, phenylalanine, arginine, cysteine and cystine; feed mixtures containing these additives and method of producing the dipeptides.

### FIELD OF THE INVENTION

The present invention relates to new methionine-bound non-natural and natural dipeptides of essential, limiting amino acids such as lysine, threonine and tryptophan, the sulphur-containing amino acids cysteine and cystine, and their synthesis and use as feed additives for feeding useful animals such as chicken, pigs, ruminants, but also in particular fish and Crustacea in aquaculture.

### BACKGROUND OF THE INVENTION

The essential amino acids (EAAs) methionine, lysine, threonine, tryptophan, histidine, valine, leucine, isoleucine, phenylalanine and arginine, and the two sulphur-containing amino acids cysteine and cystine are very important constituents of animal feed and play an important role in the economic rearing of useful animals such as chicken, pigs and ruminants. In particular, optimum distribution and sufficient supply of EAAs are decisive. As feed from natural protein sources, e.g. soya, maize and wheat, is generally deficient in certain EAAs, special supplementation with synthetic EAAs, for example DL-methionine, L-lysine, L-threonine or L-tryptophan on the one hand permits faster growth of the animals or a higher milk yield from high-yielding dairy cows, and on the other hand also more efficient utilization of the total feed. This offers a considerable economic advantage. The markets for feed additives are of considerable industrial and economic importance. In addition they are strong growth markets, attributable not least to the increasing importance of countries such as China and India.

For many animal species L-methionine ((S)-2-amino-4-methylthiobutyric acid) represents the first limiting amino acid of all the EAAs and therefore has one of the most important roles in animal nutrition and as feed additive (Rosenberg et al., J. Agr. Food Chem. 1957, 5, 694-700). In the classical chemical synthesis, however, methionine is formed as a racemate, a 50:50 mixture of D- and L-methionine. This racemic DL-methionine can, however, be used directly as feed additive, because in some animal species under in vivo conditions there is a conversion mechanism that transforms the non-natural D-enantiomer of methionine into the natural L-enantiomer. The D-methionine is first deaminated by means of a nonspecific D-oxidase to α-keto-methionine and then converted by an L-transaminase to L-methionine (Baker, D. H. in “Amino acids in farm animal nutrition”, D'Mello, J. P. F. (ed.), Wallingford (UK), CAB International, 1994, 37-61). As a result the available amount of L-methionine in the body is increased, and can then be available to the animal for growth. The enzymatic conversion of D- to L-methionine has been found in chicken, pigs and cows, but also in particular in fish, shrimp and prawns. For example, Sveier et al. (Aquacult. Nutr. 2001, 7 (3), 169-181) and Kim et al. (Aquaculture 1992, 101 (1-2), 95-103) showed that the conversion of D- to L-methionine is possible in carnivorous Atlantic salmon and rainbow trout. The same was shown by Robinson et al. (J. Nutr. 1978, 108 (12), 1932-1936) and Schwarz et al. (Aquaculture 1998, 161, 121-129) for omnivorous fish species, for example catfish and carp. Furthermore, Forster and Dominy (J. World Aquacult. Soc. 2006, 37 (4), 474-480) were able to show, in feeding tests with omnivorous shrimps of the species Litopenaeus vannamei, that DL-methionine is equally as effective as L-methionine. In the year 2007, world-wide more than 70000 tonnes of crystalline DL-methionine or racemic, liquid methionine-hydroxy-analogue (MHA, rac-2-hydroxy-4-(methylthio)butanoic acid (HMB)) and solid calcium-MHA were produced and successfully used directly as feed additive for monogastric animals, e.g. poultry and pigs.

In contrast to methionine, with lysine, threonine and tryptophan in each case only the L-enantiomers can be used as feed additives, as the respective D-enantiomers of these three essential and limiting amino acids cannot be converted by the body under physiological conditions to the corresponding L-enantiomers. Thus, the world market for L-lysine alone, the first-limiting amino acid for example for pigs, for the year 2007 was over one million tonnes. For the other two limiting essential amino acids L-threonine and L-tryptophan the world market in 2007 was over 100 000 t and just under 3000 t, respectively.

In the case of monogastric animals, e.g. poultry and pigs, usually DL-methionine, MHA, but also L-lysine, L-threonine and L-tryptophan are used directly as feed additive. In contrast, supplementation of feed with EAAs such as methionine, lysine, threonine or also MHA is not effective for ruminants, as most is broken down by microbes in the rumen of ruminants. Owing to this degradation, only a fraction of the supplemented EAAs enters the animal's small intestine, where absorption into the blood generally takes place. Among the EAAs, mainly methionine plays a decisive role in ruminants, as a high milk yield is only ensured with optimum supply. For methionine to be available to the ruminant at high efficiency, it is necessary to use a rumen-resistant protected form. There are several possible ways of imparting these properties to DL-methionine or rac-MHA. One possibility is to achieve high rumen resistance by applying a suitable protective layer or by distributing the methionine in a protective matrix. As a result methionine can pass through the rumen practically without loss. Subsequently, the protective layer is then removed e.g. in the abomasum by acid hydrolysis and the methionine that is released can then be absorbed in the small intestine of the ruminant. Commercially available products are e.g. Mepron® from the company Evonik Degussa and Smartamine™ from the company Adisseo. The production and/or coating of methionine are generally a technically complicated and laborious process and are therefore expensive. In addition, the surface coating of the finished pellets can easily be damaged by mechanical stresses and abrasion during processing of the feed, which can lead to reduction or even to complete loss of protection. Therefore it is also not possible to process the protected methionine pellets into a larger mixed-feed pellet, because once again the protecting layer would be broken up by the mechanical loading. This limits the use of such products. Another possibility for increasing rumen stability is chemical derivatization of methionine or MHA. In this, the functional groups of the molecule are derivatised with suitable protecting groups. This can be achieved e.g. by esterification of the carboxylic acid function with alcohols. As a result, degradation in the rumen by microorganisms can be reduced. A commercially available product with chemical protection is for example Metasmart™, the racemic iso-propyl ester of MHA (HMBi). A bioavailability of at least 50% for HMBi in ruminants was disclosed in WO00/28835. The chemical derivatization of methionine or MHA often has the disadvantage of poorer bioavailability and comparatively low content of active substance.

In addition to the problems of ruminal degradation of supplemented EAAs such as methionine, lysine or threonine in ruminants, there can also be various problems in fish and Crustacea in supplementation of feed with EAAs. Owing to the rapid economic development of the breeding of fish and Crustacea in highly industrialized aquaculture, means for optimum, economic and efficient supplementation of essential and limiting amino acids have become increasingly important in this particular area (Food and Agriculture Organization of the United Nations (FAO) Fisheries Department “State of World Aquaculture 2006”, 2006, Rome. International Food Policy Research Institute (IFPRI) “Fish 2020: Supply and Demand in Changing Markets”, 2003, Washington, D.C.). However, in contrast to chicken and pigs, various problems may arise when using crystalline EAAs as feed additive for certain varieties of fish and Crustacea. Thus, Rumsey and Ketola (J. Fish. Res. Bd. Can. 1975, 32, 422-426), report that the use of soya flour in conjunction with individually supplemented, crystalline amino acids did not lead to any increase in growth in the case of rainbow trout. Murai et al. (Bull. Japan. Soc. Sci. Fish. 1984, 50 (11), 1957) were able to show that the daily feeding of fish diets with high proportions of supplemented, crystalline amino acids had the result, in carp, that more than 40% of the free amino acids are excreted via the gills and kidneys. Owing to the rapid absorption of supplemented amino acids shortly after food intake, there is a very rapid rise in the concentration of amino acids in the blood plasma of the fish (fast-response). At this time, however, the other amino acids from natural protein sources, e.g. soya flour, are not yet in the plasma, which can lead to asynchronism of the simultaneous availability of all important amino acids. A proportion of the highly concentrated amino acids is in consequence rapidly excreted or quickly metabolized in the body and utilized e.g. purely as an energy source. Accordingly, in carp there is little if any increase in growth when crystalline amino acids are used as feed additives (Aoe et al., Bull. Japa. Soc. Sci. Fish. 1970, 36, 407-413). In the case of Crustacea the supplementation of crystalline EAAs can also lead to other problems. Because of the slow feeding behaviour of certain Crustacea, e.g. shrimps of the species Litopenaeus vannamei, the long time that the feed remains under water results in leaching of the supplemented, water-soluble EAAs, which leads to eutrophication of the water, instead of an increase in growth of the animals (Alam et al., Aquaculture 2005, 248, 13-16). Effective supply for fish and Crustacea in aquaculture therefore requires, for certain species and applications, a special product form of EAAs, for example an appropriately chemically or physically protected form. The aim is, firstly, that the product should remain sufficiently stable during feeding in the aqueous environment and not be leached out of the feed; and secondly, that the amino acid product finally taken in by the animal should be able to be utilized optimally and at high efficiency in the animal organism.

In the past, much effort was expended in developing suitable feed additives, especially based on the essential amino acids methionine and lysine, for fish and Crustacea. For example, WO8906497 describes the use of di- and tripeptides as feed additive for fish and Crustacea. The intention was to promote growth of the animals. However, preference was given to the use of di- and tripeptides from non-essential as well as non-limiting amino acids, e.g. glycine, alanine and serine, which are more than adequately present in many plant protein sources. Only DL-alanyl-DL-methionine and DL-methionyl-DL-glycine were described as methionine-containing dipeptides. This means, however, that the dipeptide effectively only contains 50% active substance (mol/mol), which from the economic standpoint is to be regarded as very unfavourable. WO02088667 describes the enantioselective synthesis and use of oligomers from MHA and amino acids, e.g. methionine, as feed additives, for fish and Crustacea, among others. This ought to result in faster growth. The oligomers described are formed by an enzyme-catalysed reaction and have a very wide distribution of chain length of the individual oligomers. As a result the method is non-selective, expensive and laborious in execution and purification. Dabrowski et al. describe in US20030099689 the use of synthetic peptides as feed additives for promoting the growth of aquatic animals. In this case the peptides can represent a proportion by weight of 6-50% of the total feed formulation. The synthetic peptides preferably consist of EAAs. The enantioselective synthesis of these synthetic oligo- and polypeptides is, however, very laborious, expensive and is difficult to scale up. In addition, the effectiveness of polypeptides of a single amino acid is disputed, because often they are only converted to free amino acids very slowly, or not at all, under physiological conditions. For example, Baker et al. (J. Nutr. 1982, 112, 1130-1132) show that because it is completely insoluble in water, poly-L-methionine has no bioavailability in chicken, as it cannot be absorbed by the body.

As well as the use of new chemical derivatives of EAAs such as methionine-containing peptides and oligomers, various physical means of protection, e.g. coatings or embedding an EAA in a protective matrix, have been investigated. For example, Alam et al. (Aquacult. Nutr. 2004, 10, 309-316 and Aquaculture 2005, 248, 13-19) showed that coated methionine and lysine, in contrast to uncoated products, have a very beneficial influence on the growth of young kuruma shrimps. Although the use of a special coating was able to prevent the leaching of methionine and lysine from the feed pellet, it has some serious drawbacks. The production and coating of amino acids is generally a technically complicated and laborious process, and is therefore expensive. In addition, the surface coating of the finished coated amino acid can easily be damaged by mechanical stresses and abrasion during feed processing, which can lead to reduction or even to complete loss of physical protection. Furthermore, a coating or the use of a matrix substance lowers the content of amino acid so that it often becomes uneconomic.

### SUMMARY OF THE INVENTION

A general problem was to provide a feed or a feed additive for animal nutrition based on a novel methionine-containing substitute, in which methionine is bound covalently to an essential and limiting amino acid, e.g. L-lysine, L-threonine and L-tryptophan, and which can be used as feed additives for feeding useful animals such as chicken, pigs, ruminants, though in particular also fish and Crustacea in aquaculture.

Against the background of the disadvantages of the prior art, the problem was mainly to provide a chemically protected product from the covalently bound combination of DL-methionine plus EAA such as e.g. L-lysine, L-threonine or L-tryptophan for various useful animals such as chicken, pigs and ruminants, but also for many omnivorous, herbivorous and carnivorous species of fish and Crustacea, which live in salt water or fresh water. As well as its function as a source of methionine, said product should also function as a source of all other EAAs. In particular said product should possess a “slow-release” mechanism, and thus provide slow and continuous release of free methionine and EAAs under physiological conditions. In addition, the chemically protected form of the product consisting of methionine and EAA should be rumen-resistant and so should be suitable for all ruminants. For application as feed additive for fish and Crustacea the form of the product should have low tendency to leaching from the total feed pellet or extrudate in water.

Another problem was to find a substitute for crystalline EAAs as feed or as a feed additive with very high bioavailability, which should have good handling properties and storage capability and stability under the usual conditions of mixed feed processing, in particular pelletization and extrusion.

In this way, for example chicken, pigs, ruminants, fish and Crustacea should be provided with crystalline EAAs and with other efficient sources of essential amino acids, as far as possible without the disadvantages of the known products or only having them to a reduced extent.

Furthermore, various novel and flexible synthesis routes should be developed for dipeptides containing only one methionine residue, in particular for L-EAA-DL-methionine (I) and DL-methionyl-L-EAA (II). Typical precursors and by-products from the commercial DL-methionine production process should be used as starting material for a synthetic route.

### BRIEF DESCRIPTION OF THE DRAWINGS

FIG. 1 shows the cleavage of L-EAA-L-Met (LL-I) dipeptides with enzymes from mirror carp.

FIG. 2 shows the cleavage of L-Met-L-EAA (LL-II) dipeptides with enzymes from mirror carp.

FIG. 3 shows the cleavage of L-EAA-L-Met (LL-I) dipeptides with enzymes from rainbow trout.

FIG. 4 shows the cleavage of L-Met-L-EAA (LL-II) dipeptides with enzymes from rainbow trout.

FIG. 5 shows the cleavage of L-EAA-L-Met (LL-I) dipeptides with enzymes from whiteleg shrimps.

FIG. 6 shows the cleavage of L-Met-L-EAA (LL-II) dipeptides with enzymes from whiteleg shrimps.

FIG. 7 shows the cleavage of L-EAA-D-Met (LD-I) and D-Met-L-EAA (DL-II) dipeptides with enzymes from mirror carp.

FIG. 8 shows the cleavage of L-EAA-D-Met (LD-I) and D-Met-L-EAA (DL-II) dipeptides with enzymes from grass carp.

FIG. 9 shows the cleavage of L-EAA-D-Met (LD-I) and D-Met-L-EAA (DL-II) dipeptides with enzymes from Tilapia.

FIG. 10 shows the cleavage of L-EAA-D-Met (LD-I) and D-Met-L-EAA (DL-II) dipeptides with enzymes from whiteleg shrimps.

FIG. 11 shows the cleavage of L-EAA-D-Met (LD-I) and D-Met-L-EAA (DL-II) dipeptides with enzymes from rainbow trout.

FIG. 12 shows the cleavage of mixtures of L-Trp-D-Met/D-Met-L-Trp (LD-Ij/DL-IIj) and L-Trp-D-Met/L-Met-L-Trp (LD-Ij/LL-IIj) with enzymes from mirror carp.

FIG. 13 shows the in vitro cleavage of the natural L-Ile-L-Met (LL-Ic) or L-Met-L-Ile (LL-IIc) dipeptides with 1% enzyme solution and the non-natural L-Ile-D-Met (LD-Ic) or D-Met-L-Ile (DL-IIc) dipeptides with 10% enzyme solution from mirror carp.

FIG. 14 shows the in vitro cleavage of the natural L-Thr-L-Met (LL-Id) or L-Met-L-Thr (LL-IId) dipeptides with 1% enzyme solution and the non-natural L-Thr-D-Met (LD-Id) or D-Met-L-Thr (DL-IId) dipeptides with 10% enzyme solution from mirror carp.

FIG. 15 shows the in vitro cleavage of the natural L-Lys-L-Met (LL-Ie) or L-Met-L-Lys (LL-IIe) dipeptides with 1% enzyme solution and the non-natural L-Lys-D-Met (LD-Ie) or D-Met-L-Lys (DL-IIe) dipeptides with 10% enzyme solution from mirror carp.

FIG. 16 shows the cleavage of L-Met-L-EAA (LL-II) dipeptides with enzymes from chicken.

FIG. 17 shows the cleavage of L-EAA-D-Met (LD-I) and D-Met-L-EAA (DL-II) dipeptides with enzymes from chicken.

### DETAILED DESCRIPTION OF THE INVENTION

The problem is solved with feed additives containing dipeptides or salts thereof, where one amino acid residue of the dipeptide is a DL-methionyl residue and the other amino acid residue of the dipeptide is an amino acid in the L-configuration selected from the group comprising lysine, threonine, tryptophan, histidine, valine, leucine, isoleucine, phenylalanine, arginine, cysteine and cystine.

Preferably the feed additive contains dipeptides of general formula DL-methionyl-L-EAA (=mixture of D-methionyl-L-EAA and L-methionyl-L-EAA) and/or L-EAA-DL-methionine (=mixture of L-EAA-D-methionine and L-EAA-L-methionine), where L-EAA is an amino acid in the L-configuration selected from the group comprising lysine, threonine, tryptophan, histidine, valine, leucine, isoleucine, phenylalanine, arginine, cysteine and cystine.

The invention further relates to a feed mixture containing said feed additive.

The feed additive containing L-EAA-DL-methionine and/or DL-methionyl-L-EAA and salts thereof is suitable as feed additive in feed mixtures for poultry, pigs, ruminants, but also in particular for fish and Crustacea in aquaculture.

Preferably the feed mixture contains 0.01 to 5 wt. %, preferably 0.05 to 0.5 wt. % L-EAA-DL-methionine and DL-methionyl-L-EAA.

The use of L-EAA-DL-methionine and DL-methionyl-L-EAA has proved to be particularly advantageous, because these dipeptides have good leaching behaviour owing to the low solubility.

Furthermore, the compound displays good pelletization and extrusion stability in feed production. The dipeptides L-EAA-DL-methionine and DL-methionyl-L-EAA are stable in mixtures with the usual components and feeds e.g. cereals (e.g. maize, wheat, triticale, barley, millet, etc.), plant or animal protein carriers (e.g. soya beans and rape and products from their further processing, legumes (e.g. peas, beans, lupins, etc.), fish-meal, etc.) and in combination with supplemented essential amino acids, proteins, peptides, carbohydrates, vitamins, minerals, fats and oils.

A further advantage is that because of the high proportion of active substance of L-EAA-DL-methionine and DL-methionyl-L-EAA per kg of substance, compared with DL-methionine and L-EAA, one mole of water is saved per mole of L-EAA-DL-methionine or DL-methionyl-L-EAA.

In a preferred use, the feed mixture contains proteins and carbohydrates, preferably based on fish-meal, soya flour or maize flour, and can be supplemented with essential amino acids, proteins, peptides, vitamins, minerals, carbohydrates, fats and oils.

In particular, it is preferable for the DL-methionyl-L-EAA and L-EAA-DL-methionine to be present in the feed mixture alone as D-methionyl-L-EAA, L-methionyl-L-EAA, L-EAA-D-methionine or L-EAA-L-methionine, as a mixture with one another or also as a mixture with D-methionyl-D-EAA, L-methionyl-D-EAA, D-EAA-D-methionine or D-EAA-L-methionine, preferably in each case additionally mixed with DL-methionine, preferably with a proportion of DL-methionine from 0.01 to 90 wt. %, preferably from 0.1 to 50 wt. %, especially preferably from 1 to 30 wt. %, preferably in each case additionally mixed with an L-EAA, for example L-lysine, preferably with a proportion of L-EAA from 0.01 to 90 wt. %, preferably from 0.1 to 50 wt. %, especially preferably from 1 to 30 wt. %.

In a preferred use, the animals kept in aquaculture are fresh-water and seawater fishes and Crustacea selected from the group comprising carp, trout, salmon, catfish, perch, flatfish, sturgeon, tuna, eels, bream, cod, shrimps, krill and prawns, quite especially silver carp (Hypophthalmichthys molitrix), grass carp (Ctenopharyngodon idella), scaly carp (Cyprinus carpio) and bighead carp (Aristichthys nobilis), crucian carp (Carassius carassius), catla (Catla catla), roho labeo (Labeo rohita), Pacific and Atlantic salmon (Salmo salar and Oncorhynchus kisutch), rainbow trout (Oncorhynchus mykiss), American catfish (Ictalurus punctatus), African catfish (Clarias gariepinus), pangasius (Pangasius bocourti and Pangasius hypothalamus), Nile tilapia (Oreochromis niloticus), milkfish (Chanos chanos), cobia (Rachycentron canadum), whiteleg shrimp (Litopenaeus vannamei), black tiger shrimp (Penaeus monodon) and giant river prawn (Macrobrachium rosenbergii).

According to the invention, L-EAA-DL-methionine (L-EAA-DL-Met) (I) and DL-methionyl-L-EAA (DL-Met-L-EAA) (II) or alkali and alkaline-earth salts thereof, e.g. the sparingly soluble calcium or zinc salts, are used as additive in feed mixtures as D-methionyl-L-EAA, L-methionyl-L-EAA, L-EAA-D-methionine or L-EAA-L-methionine or in the respective diastereomeric mixtures, alone or mixed with DL-methionine, alone or mixed with L-EAA preferably for poultry, pigs, ruminants, and especially preferably for fish and Crustacea:

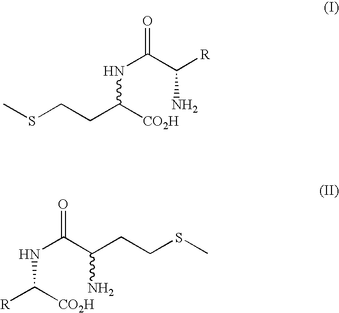

L-EAA-DL-methionine (I) has the two diastereomers L-EAA-D-Met (LD-I) and L-EAA-L-Met (LL-I). Similarly, the dipeptide DL-methionyl-L-EAA (II) has the two different stereoisomers D-Met-L-EAA (DL-II) and L-Met-L-EAA (LL-II). Only the two diastereomers L-EAA-L-Met (LL-I) and L-Met-L-EAA (LL-II) are natural, but the other two L-EAA-D-Met (LD-I) and D-Met-L-EAA (DL-II) are non-natural (see Scheme 1).

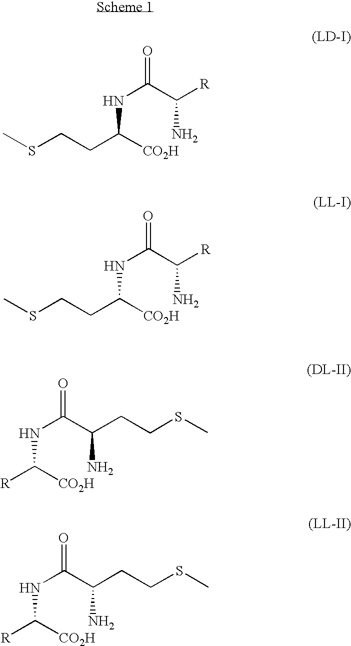

In the above, the residue R of EAA stands for:

| | | |
|---|---|---|
| Ia or IIa: | R = 1-methylethyl- | (valine) |
| Ib or IIb: | R = 2-methylpropyl- | (leucine) |
| Ic or IIc | R = (1S)-1-methylpropyl- | (isoleucine) |
| Id or IId | R = (1R)-1-hydroxyethyl- | (threonine) |
| Ie or IIe | R = 4-aminobutyl- | (lysine) |
| If or IIf | R = 3-[(aminoiminomethyl)-amino]propyl- | (arginine) |
| Ig or IIg | R = benzyl- | (phenylalanine) |
| Ih or IIh | R = (1H-imidazol-4-yl)methyl- | (histidine) |
| Ij or IIj | R = (1H-indol-3-yl)methyl- | (tryptophan) |

The stereoisomers L-EAA-D-methionine (LD-I), L-EAA-L-methionine (LL-I), D-methionyl-L-EAA (DL-II) and L-methionyl-L-EAA (LL-II) can be used as feed additive, alone or mixed with one another, preferably for poultry, pigs, ruminants, fishes, Crustacea, as well as for pets.

In addition to the development of a novel synthesis route for the preparation of L-EAA-DL-methionine (I) and DL-methionyl-L-EAA (II), the main object of the present invention is the use of I and II as diastereomeric mix from a mixture of D-methionyl-L-EAA (DL-II) and L-methionyl-L-EAA (LL-II) or from a mixture of L-EAA-D-methionine (LD-I) and L-EAA-L-methionine (LL-I) or in each case as individual diastereomer D-methionyl-L-EAA (DL-II), L-methionyl-L-EAA (LL-II), L-EAA-D-methionine (LD-I) or L-EAA-L-methionine (LL-I) as growth promoter for poultry, pigs, ruminants, but also for omnivorous, carnivorous and herbivorous fish and Crustacea in aquaculture. Moreover, by using L-EAA-DL-methionine (I) or DL-methionyl-L-EAA (II) as feed additive, the milk yield of high-yielding dairy cows can be increased.

Thus, it was shown, as an inventive step, that L-EAA-DL-methionine (I) or DL-methionyl-L-EAA (II) as a diastereomeric mix from a 50:50 mixture of L-EAA-D-methionine (LD-I) and L-EAA-L-methionine (LL-I) or from a 50:50 mixture of D-methionyl-L-EAA (DL-II) and L-methionyl-L-EAA (LL-II) or in each case as individual diastereomer can be cleaved enzymatically, under physiological conditions, by chicken, pigs, cows, fishes such as e.g. carp and trout, but also by Crustacea such as for example Litopenaeus vannamei (whiteleg shrimp) and Macrobrachium rosenbergii (giant river prawn) to free D- or L-methionine and in each case to L-EAA (see Scheme 2).

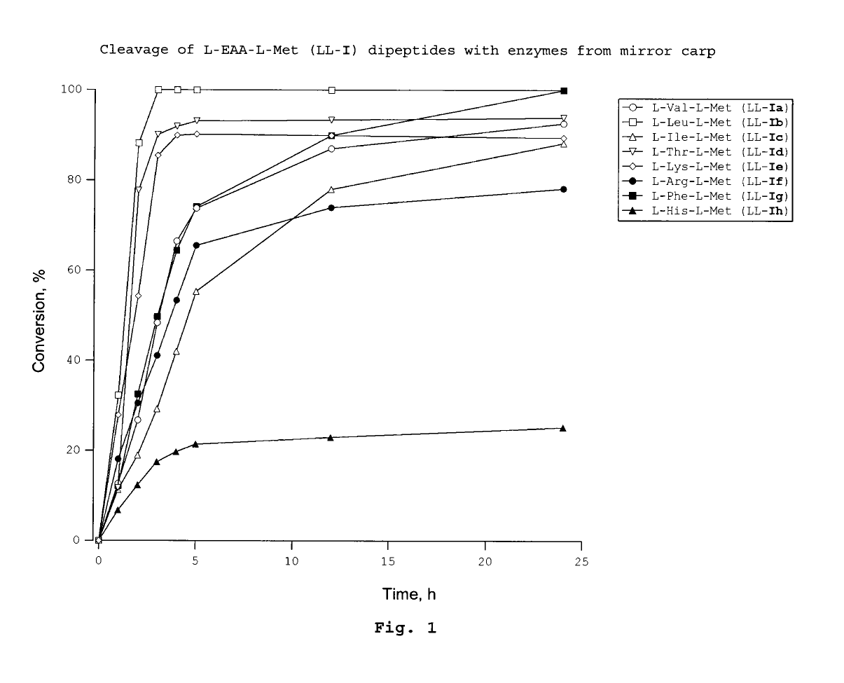

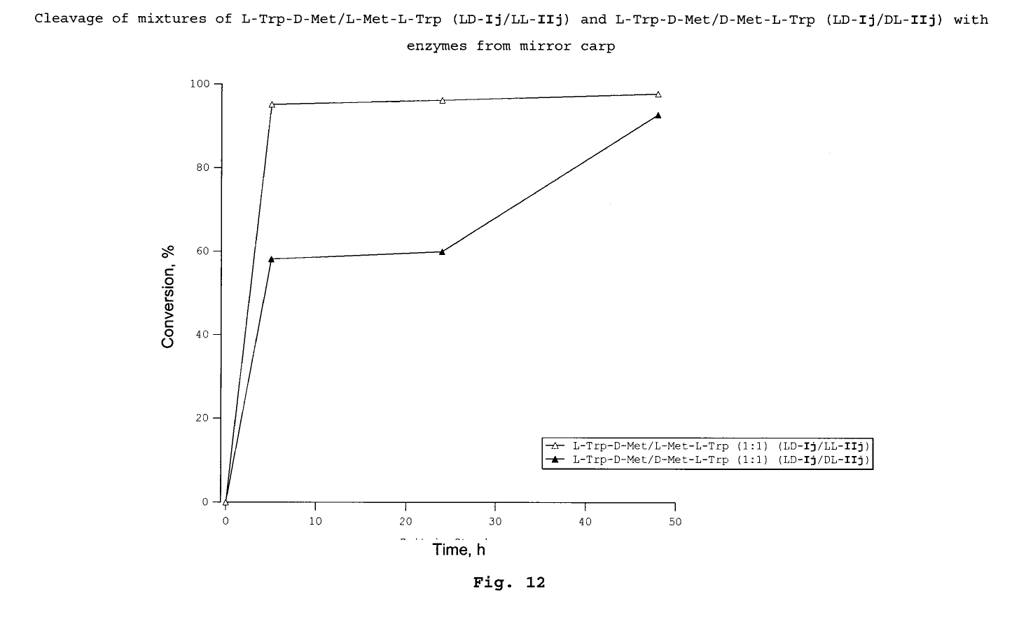

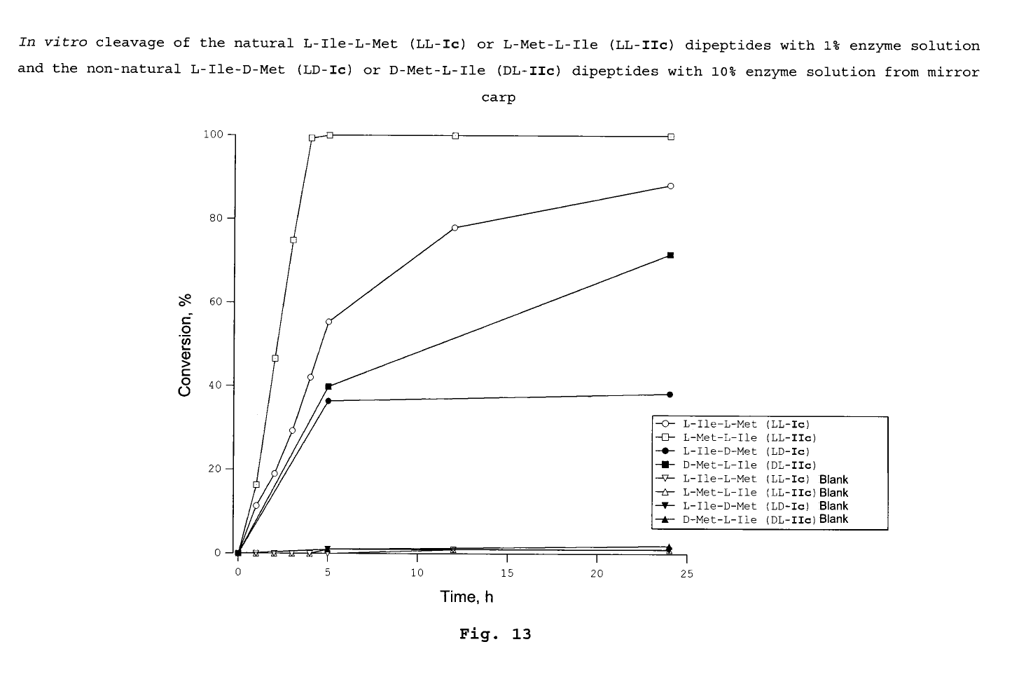

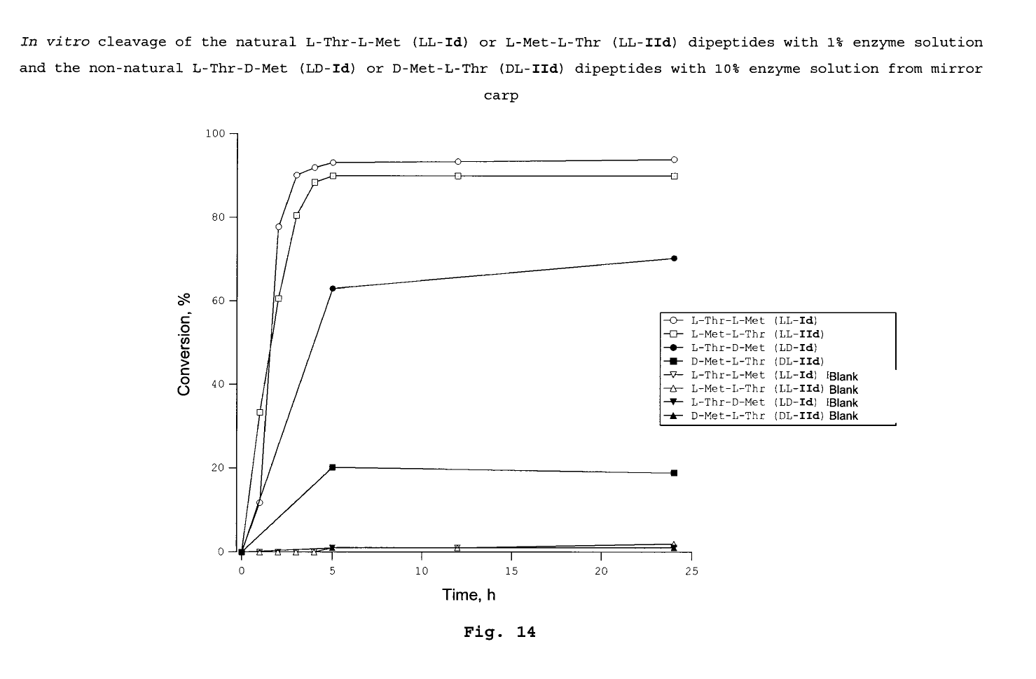

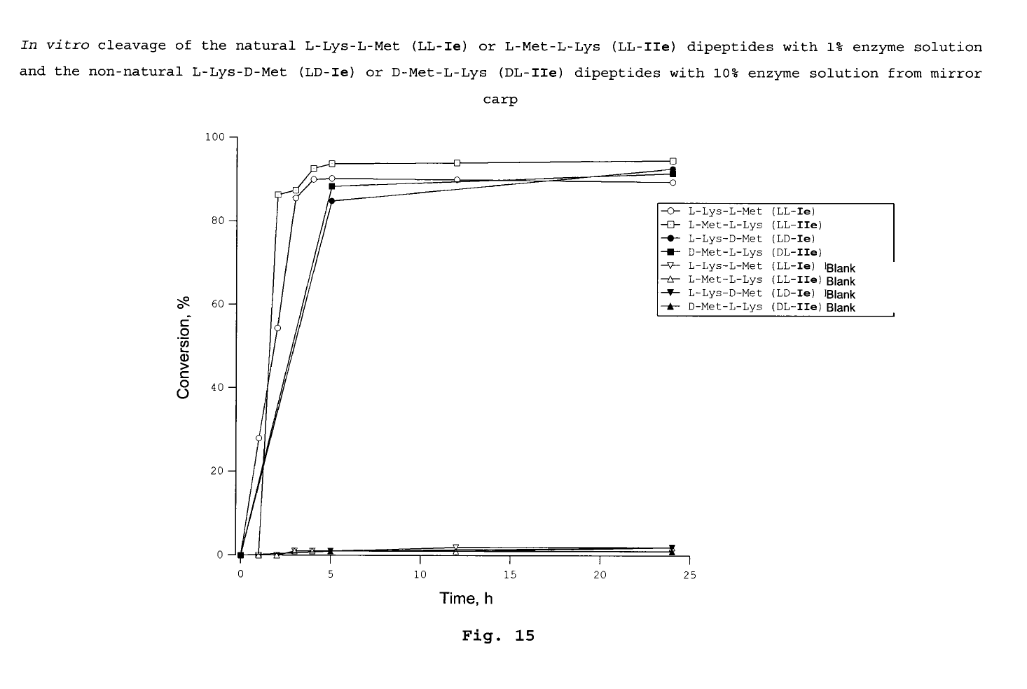

For this, the corresponding digestive enzymes were isolated for example from chicken, omnivorous carp, carnivorous trout and omnivorous whiteleg shrimps (Litopenaeus vannamei) and reacted in optimized in vitro tests under physiologically comparable conditions with DL-methionyl-L-EAA (II) as a diastereomeric mix from a 50:50 mixture of D-methionyl-L-EAA (DL-II) and L-methionyl-L-EAA (LL-II) or L-EAA-DL-methionine (I) from a 50:50 mixture of L-EAA-D-methionine (LD-I) and L-EAA-L-methionine (LL-I) or in each case as individual diastereomer D-methionyl-L-EAA (DL-II), L-methionyl-L-EAA (LL-II), L-EAA-D-methionine (LD-I) or L-EAA-L-methionine (LL-I). The special feature according to the invention of the cleavage of L-EAA-DL-methionine (I) or DL-methionyl-L-EAA (II) is that, in addition to the two natural diastereomers L-EAA-L-methionine (LL-I) and L-methionyl-L-EAA (LL-II), also the two non-natural diastereomers L-EAA-D-methionine (LD-I) and D-methionyl-L-EAA (DL-II) can be cleaved under physiological conditions (see FIGS. 1 to 17). This applies both to the use of the mixture of D-methionyl-L-EAA (DL-II) and L-methionyl-L-EAA (LL-II), the mixture of D-methionyl-L-EAA (DL-II) and L-EAA-D-methionine (LD-I) (see FIG. 12) or the mixture of L-methionyl-L-EAA (LL-II) and L-EAA-D-methionine (LD-I) (see FIG. 12), but also for the total mixture of all diastereomers, and in each case for the individual diastereomers (see FIGS. 1 to 11 and 13 to 17).

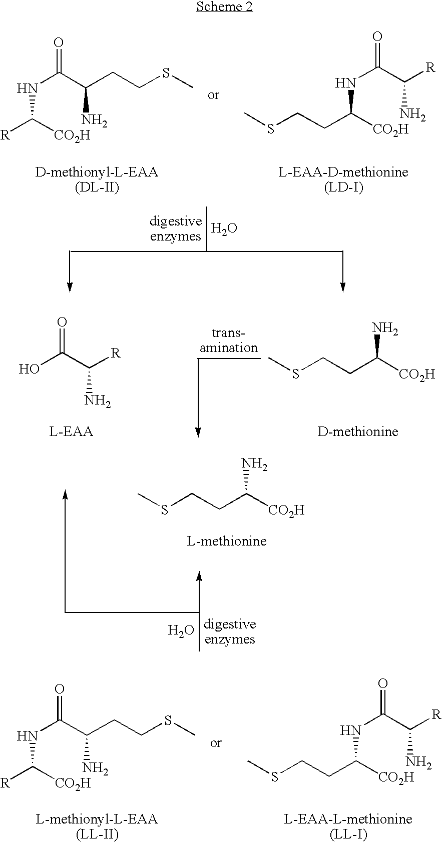

The natural dipeptides L-EAA-L-Met (LL-I) and L-Met-L-EAA (LL-II) were digested with digestive enzymes from carnivorous rainbow trout, omnivorous mirror carp, omnivorous whiteleg shrimps and chicken (see Table 1).

TABLE 1

| | Species | | | |
|---|---|---|---|---|
| | | Mirror | White-leg | |
| | Trout | carp | shrimp | Chicken |
| | (carniv- | (omniv- | (omniv- | (omniv- |
| Dipeptide | orous) | orous) | orous) | orous) |
| L-Met-L-Val (LL-IIa) | x | x | x | |
| L-Met-L-Leu (LL-IIb) | x | x | x | |
| L-Met-L-Ile (LL-IIc) | x | x | x | |
| L-Met-L-Thr (LL-IId) | x | x | x | |
| L-Met-L-Lys (LL-IIe) | x | x | x | |
| L-Met-L-Arg (LL-IIf) | x | x | x | |
| L-Met-L-Phe (LL-IIg) | x | x | x | |
| L-Met-L-His (LL-IIh) | x | x | x | |
| L-Met-L-Trp (LL-IIj) | x | x | x | |
| L-Val-L-Met (LL-Ia) | x | x | x | |
| L-Leu-L-Met (LL-Ib) | x | x | x | |
| L-Ile-L-Met (LL-Ic) | x | x | x | |
| L-Thr-L-Met (LL-Id) | x | x | x | x |
| L-Lys-L-Met (LL-Ie) | x | x | x | x |
| L-Arg-L-Met (LL-If) | x | x | x | |
| L-Phe-L-Met (LL-Ig) | x | x | x | |
| L-His-L-Met (LL-Ih) | x | x | x | |
| L-Trp-L-Met (LL-Ij) | x | x | x | |

For this, the enzymes were separated from the digestive tracts of the fishes and shrimps. The dipeptides L-EAA-L-Met (LL-I) and L-Met-L-EAA (LL-II) would then be digested with the enzyme solutions obtained. For better comparability of the digestibilities of dipeptides of different species, identical conditions were selected for the in vitro digestion studies (37° C., pH 9).

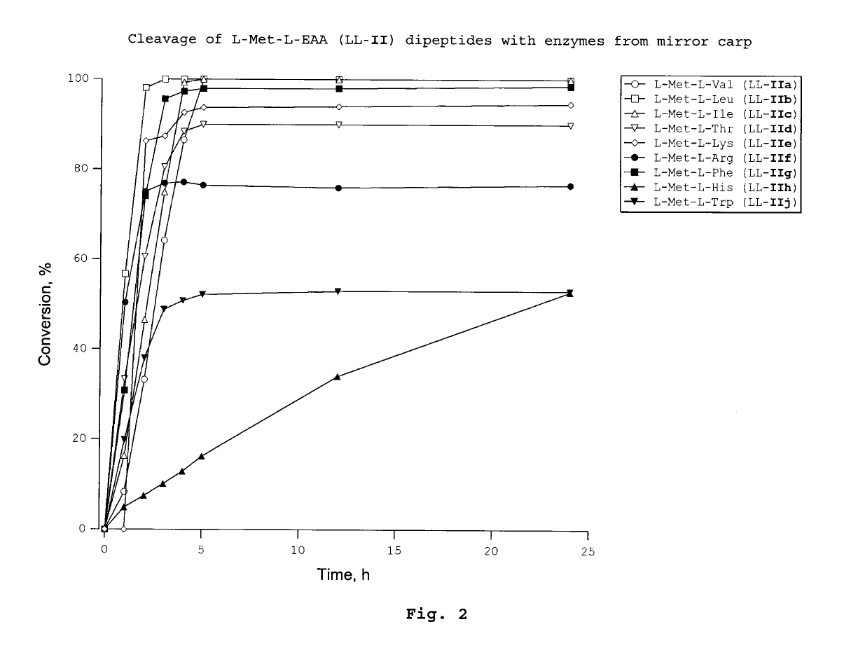

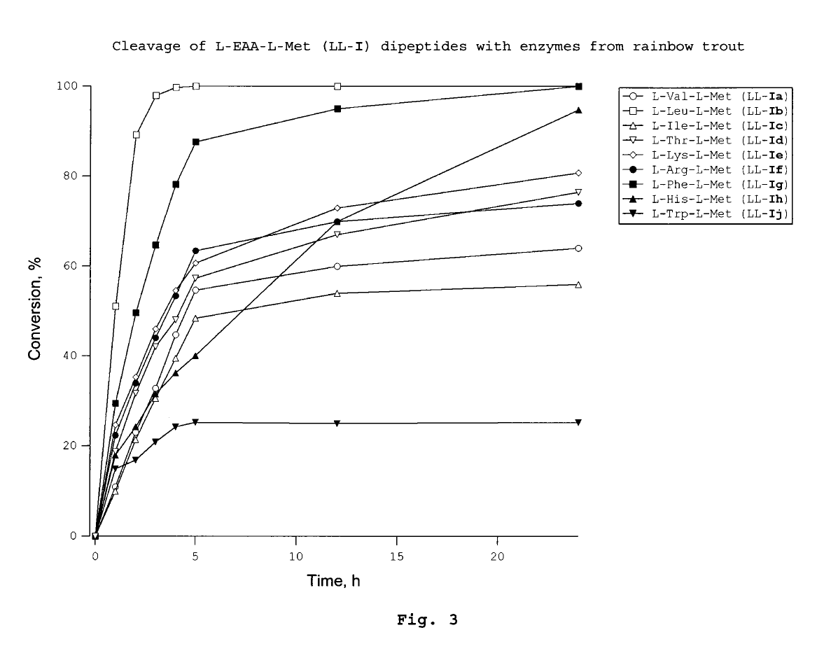

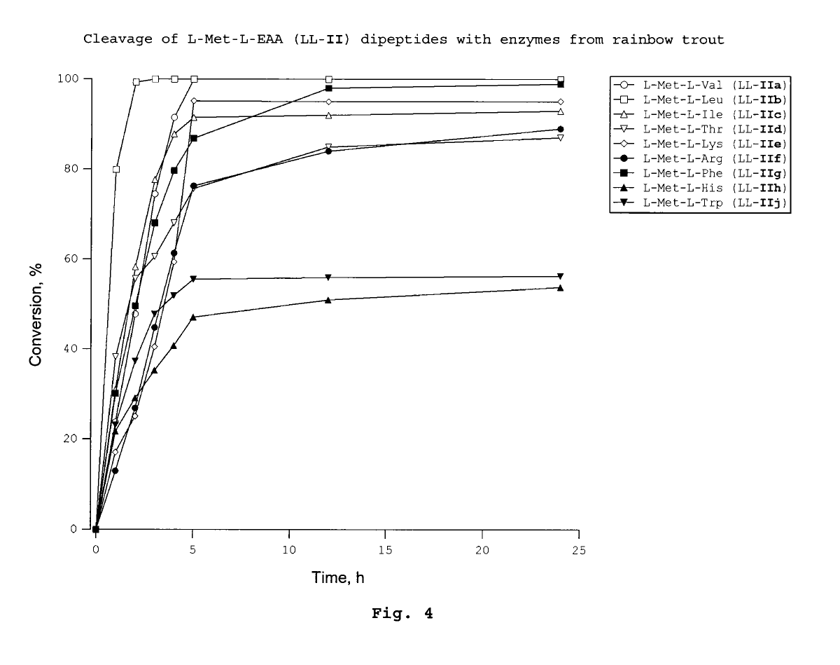

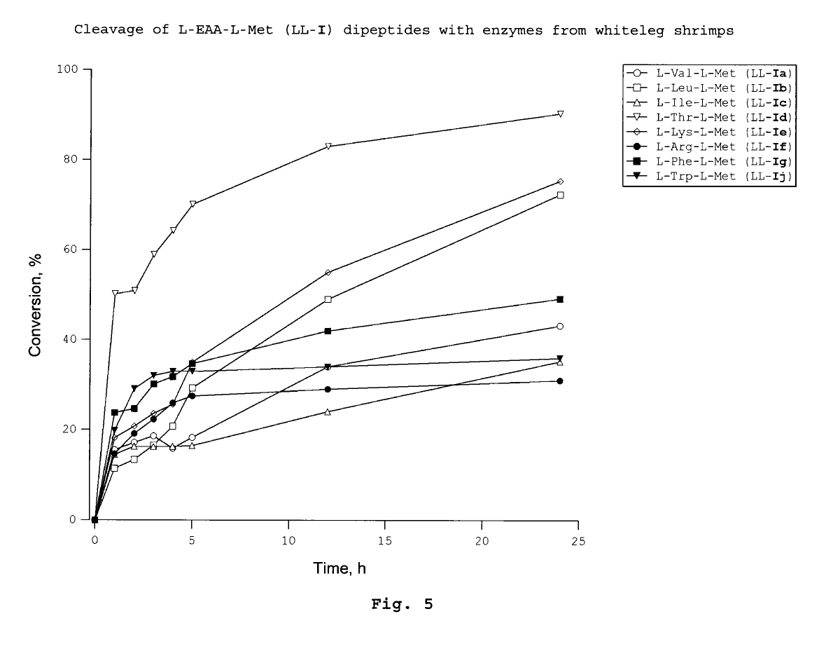

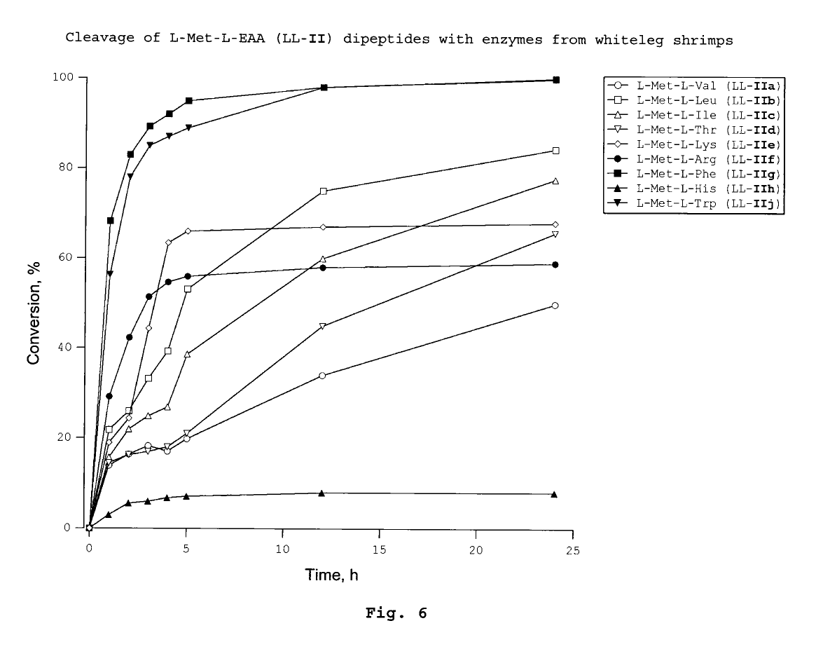

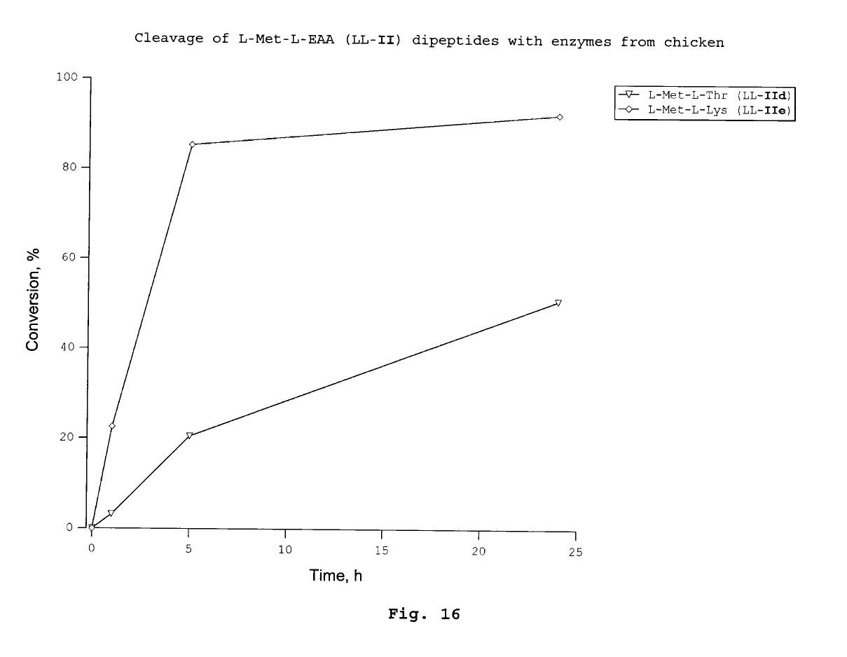

All natural dipeptides are cleaved by digestive enzymes of the carnivorous rainbow trout (see FIGS. 3 and 4), of the omnivorous mirror carp (see FIGS. 1 and 2), of the omnivorous whiteleg shrimps (see FIGS. 5 and 6) and of the chicken (see FIG. 16). Cleavages of L-Met-L-EAA (LL-II) as a rule proceed more quickly than the cleavages of the analogous L-EAA-L-Met (LL-I) dipeptides.

In order to demonstrate the enzymatic cleavage of non-natural dipeptides L-EAA-D-Met (LD-I) and D-Met-L-EAA (DL-II) by digestive enzymes of various fish species as comprehensively as possible, an experimental matrix was investigated (see Table 2).

TABLE 2

| | Dipeptide | | | | | | | | | | | |
|---|---|---|---|---|---|---|---|---|---|---|---|---|
| | D-Met- | | D- | D-Met- | D-Met- | D-Met- | L-Trp- | L-hr- | L-Lys- | L-Leu- | L-Ile- | L-Phe- |
| | L-Trp | D-Met-L-Thr | Met-L-Lys | L-Leu | L-Ile | L-Phe | D-Met | D-Met | D-Met | D-Met | D-Met | D-Met |
| Species | (DL-IIj) | (DL-IId) | (DL-IIe) | (DL-IIb) | (DL-IIc) | (DL-IIg) | (DL-Ij) | (DL-Id) | (DL-Ie) | (DL-Ib) | (DL-Ic) | (DL-Ig) |
| Trout | x | x | x | x | | | x | x | x | x | | |
| (carnivorous) | | | | | | | | | | | | |
| Mirror carp | x | x | x | | x | | x | x | x | | x | |
| (omnivorous) | | | | | | | | | | | | |
| Grass carp | x | x | x | | | x | x | x | x | | | x |
| (herbivorous) | | | | | | | | | | | | |
| Whiteleg shrimp | x | x | x | x | | | x | x | x | x | | |
| (omnivorous) | | | | | | | | | | | | |
| Tilapia | x | | x | | | | x | | x | | | |
| (omnivorous) | | | | | | | | | | | | |
| Chicken | | x | x | | x | | | x | x | | x | |
| (omnivorous) | | | | | | | | | | | | |

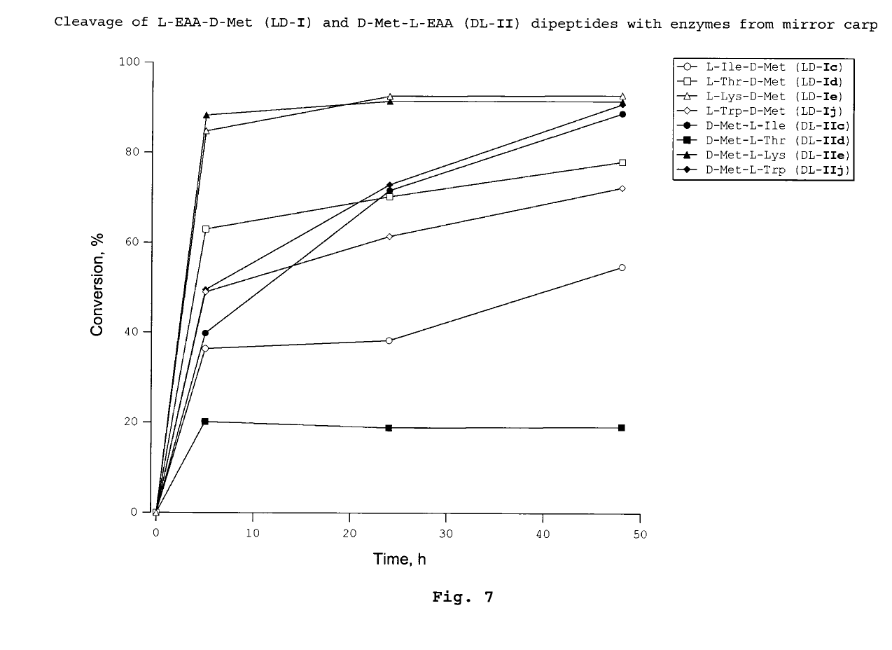

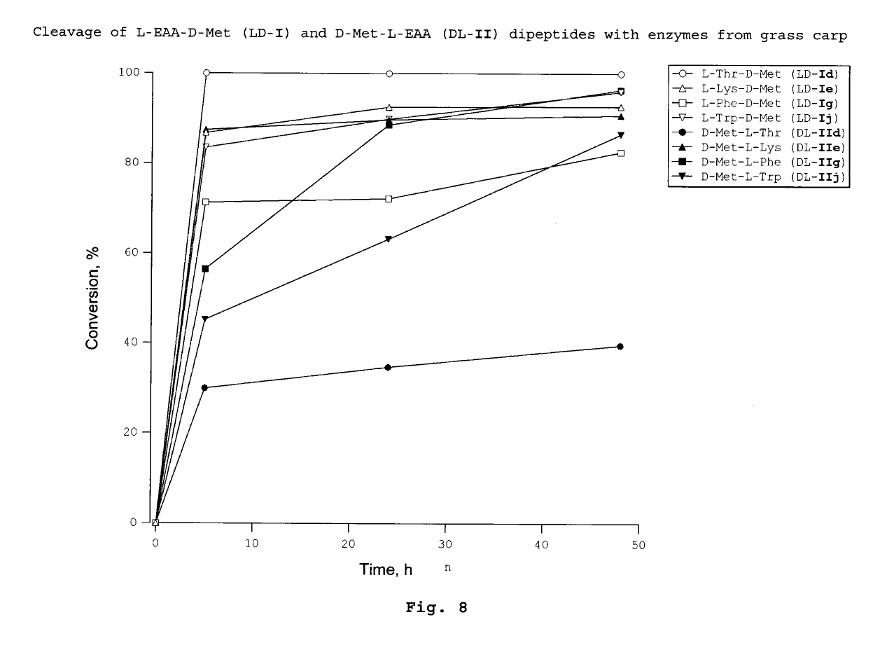

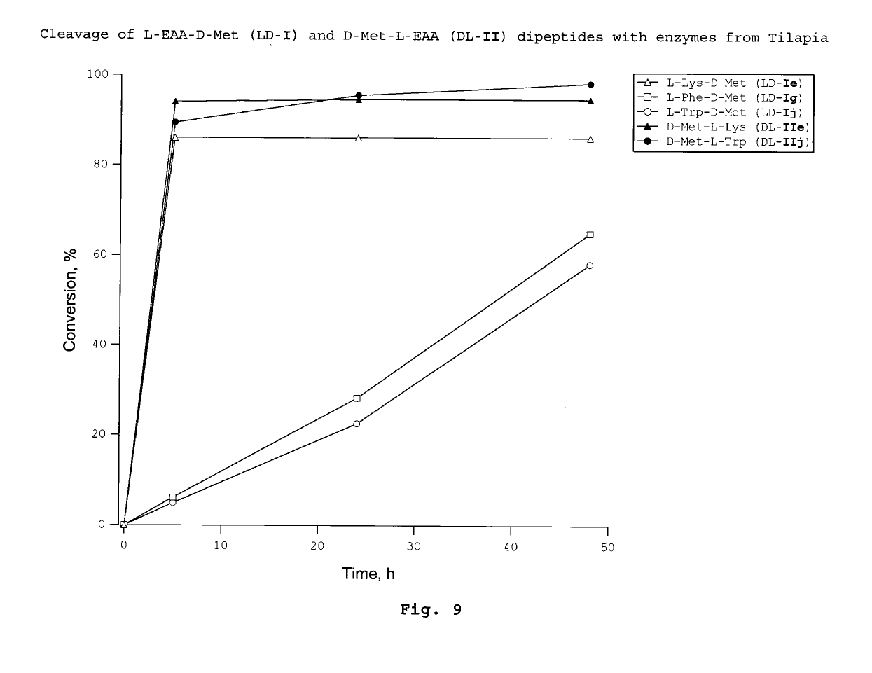

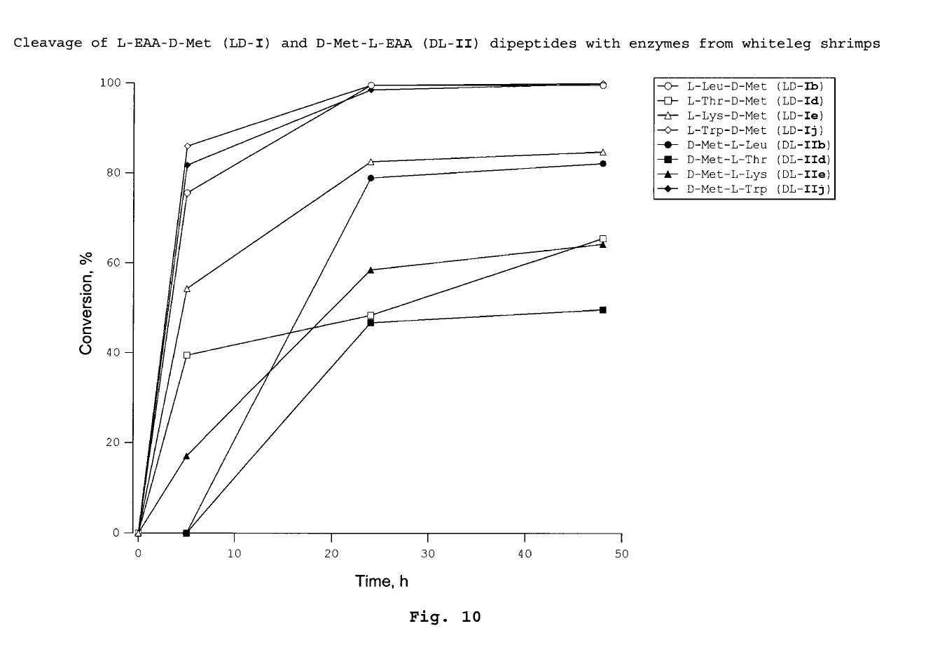

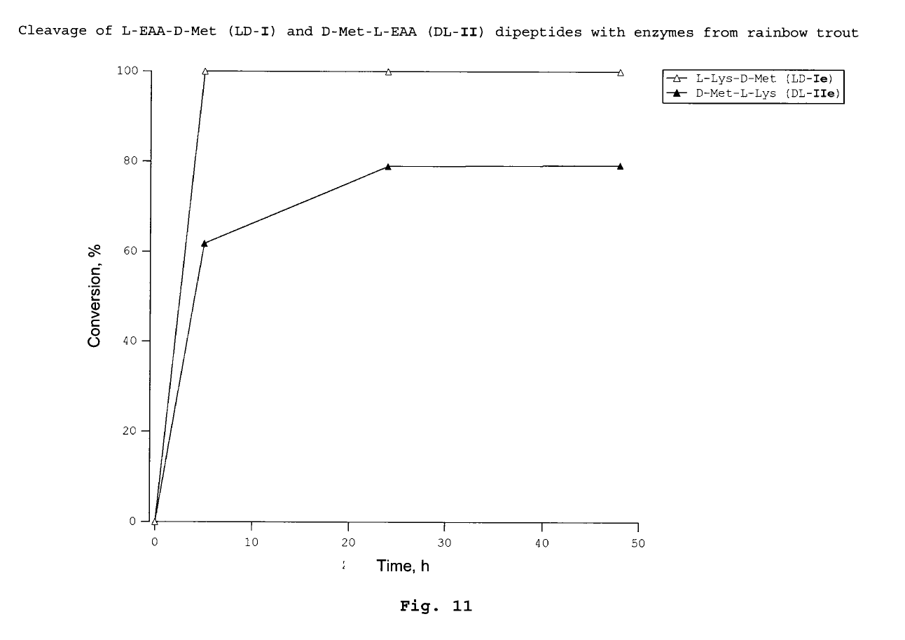

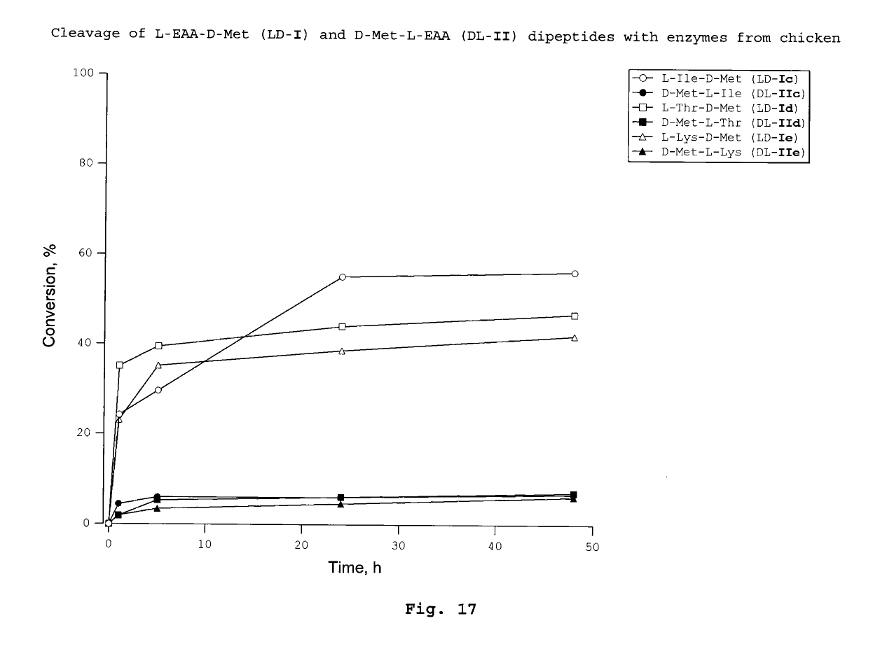

For this, the enzymes were isolated from the digestive tracts of the fishes and shrimps. The chemically synthesized dipeptides L-EAA-D-Met (LD-I) and D-Met-L-EAA (DL-II) were then reacted with the enzyme solutions obtained. For better comparability of the digestibilities of dipeptides of various species, identical conditions were selected for the in vitro digestion studies (37° C., pH 9). All non-natural dipeptides L-EAA-D-Met (LD-I) and D-Met-L-EAA (DL-II) are cleaved by digestive enzymes of the omnivorous mirror carp (see FIG. 7), of the herbivorous grass carp (see FIG. 8), of the carnivorous rainbow trout (see FIG. 11), of the omnivorous whiteleg shrimp (see FIG. 10) and of the chicken (see FIG. 17). The cleavages of D-Met-L-EAA (DL-II) proceed somewhat more slowly than the cleavages of the analogous L-EAA-D-Met (LD-I) dipeptides. With digestive enzymes of the Tilapia (see FIG. 9), in contrast, D-Met-L-EAA (DL-II) could be cleaved more quickly than L-EAA-D-Met (LD-I) dipeptides. The dipeptides D-Met-L-Lys (DL-IIe) and L-Lys-D-Met (LD-Ie) are digested particularly quickly. After just 5 hours, under in vitro reaction conditions the bulk of the lysine-containing dipeptides had been cleaved by all of the digestive enzymes used.

It follows from the results obtained that each non-natural dipeptide used (see FIGS. 7 to 11 and 17) can be cleaved with digestive enzymes of various fish species, shrimps and chicken. By using enzymes from carnivorous rainbow trout, omnivorous mirror carp, tilapias, whiteleg shrimps, herbivorous grass carp, and chicken, it was demonstrated that the non-natural dipeptides L-EAA-D-Met (LD-I) and D-Met-L-EAA (DL-II) can be cleaved in vitro by all animals, which have markedly different digestive systems. By adding L-EAA-D-Met (LD-I) and/or D-Met-L-EAA (DL-II) dipeptides to the feed, it is thus possible to supply deficient essential amino acids (DL-Met and L-EAA) as required.

The cleavage of dipeptide mixtures of natural and non-natural dipeptides was investigated for the example of dipeptides from the amino acids L-tryptophan and DL-methionine. The diastereomeric mix consisting of the two non-natural dipeptides L-Trp-D-Met (LD-Ij) and D-Met-L-Trp (DL-IIj) could be cleaved completely, just like the mixture of the natural dipeptide L-Met-L-Trp (LL-IIj) and the non-natural dipeptide L-Trp-D-Met (LD-Ij). The “slow-release” effect is much more pronounced with the LD-Ij/DL-IIj mix than with the LD-Ij/LL-IIj mix, i.e. the amino acids tryptophan and methionine are released by enzymatic digestion of the dipeptides more slowly relative to one another and over a longer period.

The problem is in addition solved with a dipeptide or a salt thereof of general formula DL-methionyl-DL-EAA or DL-EAA-DL-methionine, where EAA is an amino acid, preferably in the L-configuration selected from the group comprising lysine, threonine, tryptophan, histidine, valine, leucine, isoleucine, phenylalanine, arginine, cysteine and cystine. The methionyl residue in the D- or L-configuration is equally preferred. This includes the dipeptides Met-Lys, Met-Thr, Met-Trp, Met-His, Met-Val, Met-Leu, Met-Ile, Met-Phe, Met-Arg, Met-Cys and Met-cystine, in each case in the configurations DD, LD, DL and LL, and Lys-Met, Thr-Met, Trp-Met, His-Met, Val-Met, Leu-Met, Ile-Met, Phe-Met, Arg-Met, Cys-Met and cystine-Met, in each case in the configurations DD, LD, DL and LL.

The problem is furthermore solved by a method of production of a dipeptide containing only one methionyl residue according to the formula DD/LL/DL/LD-I or DD/LL/DL/LD-II:

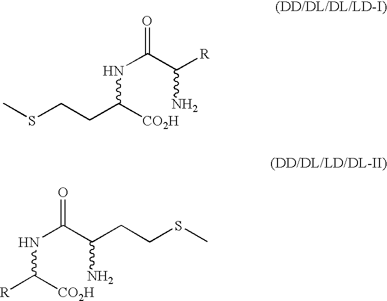

by reaction of an amino acid with a urea derivative of general formula III to V,

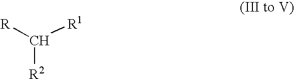

with R defined as follows:

| | | |
|---|---|---|
| Ia to Va: | R = 1-methylethyl- | (valine) |
| Ib to Vb: | R = 2-methylpropyl- | (leucine) |
| Ic to Vc: | R = (1S)-1-methylpropyl- | (isoleucine) |
| Id to Vd: | R = (1R)-1-hydroxyethyl- | (threonine) |
| Ie to Ve: | R = 4-aminobutyl- | (lysine) |
| If to Vf: | R = 3-[(aminoiminomethyl)-amino]propyl- | (arginine) |
| Ig to Vg: | R = benzyl- | (phenylalanine) |
| Ih to Vh: | R = (1H-imidazol-4-yl)methyl- | (histidine) |
| Ij to Vj: | R = (1H-indol-3-yl)methyl- | (tryptophan) |
| Ik to Vk: | R = —CH₂—SH | (cysteine) |
| Im to Vm: | R = —CH₂—S—S—CH₂—CNH₂—COOH | (cystine) |
| IIIn to Vn: | R = —CH₂—CH₂—S—CH₃ | (methionine) |

with the residues R¹ and R² in the urea derivatives III, IV and V being defined as follows:
where
IIIa-n: R¹═COOH, R²═NHCONH₂
IVa-n: R¹═CONH₂, R²═NHCONH₂
Va-n: R¹-R²=—CONHCONH—
   and
   where R either denotes a methionyl residue and the added amino acid is selected from the group comprising lysine, threonine, tryptophan, histidine, valine, leucine, isoleucine, phenylalanine, arginine, cysteine, or cystine; or
   the added amino acid is methionine and R is an amino acid residue selected from the group comprising lysine, threonine, tryptophan, histidine, valine, leucine, isoleucine, phenylalanine, arginine, cysteine, or cystine.

In a preferred embodiment, methionine hydantoin or the hydantoin of an amino acid selected from the group comprising lysine, threonine, tryptophan, histidine, valine, leucine, isoleucine, phenylalanine, arginine, cysteine, cystine is used as starting product or is formed as an intermediate.

In one embodiment of the method according to the invention it is preferred for a solution containing methionine hydantoin (Vn) and water to be reacted with the amino acid under basic conditions, or a solution containing the hydantoin of the amino acid selected from the group comprising lysine, threonine, tryptophan, histidine, valine, leucine, isoleucine, phenylalanine, arginine, cysteine, cystine and water to be reacted with methionine under basic conditions.

In another embodiment of the method according to the invention it is preferable for methionine hydantoin (Vn) to be used as starting product or to be formed as an intermediate. The preferred production of DL-methionyl-L-EAA (II) directly from methionine hydantoin (Vn), N-carbamoylmethionine (IIIn) or N-carbamoylmethioninamide (IVn) is shown in Scheme 3 and comprises method A.

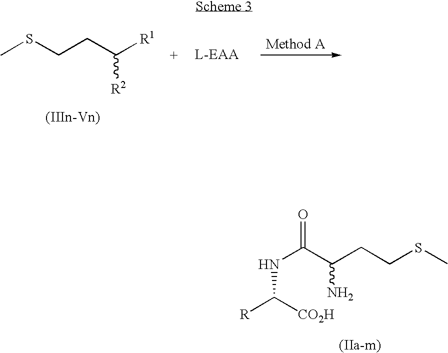

Furthermore it is preferable for the pH value of the solution containing the urea derivative to be adjusted to 7 to 14, preferably to 8 to 13 and quite especially preferably to 9 to 12.

The reaction is preferably carried out at a temperature from 30 to 200° C., preferably at a temperature from 80 to 170° C. and especially preferably at a temperature from 120 to 160° C.

Furthermore, it is preferable for the reaction to be carried out under pressure, preferably at a pressure from 2 to 100 bar, especially preferably at a pressure from 4 to 60 bar, quite especially preferably at a pressure from 8 to 40 bar.

In another preferred method the solution containing methionine hydantoin and water or the solution containing hydantoin of the amino acid selected from the group comprising lysine, threonine, tryptophan, histidine, valine, leucine, isoleucine, phenylalanine, arginine, cysteine, cystine and water was formed beforehand from one or more of the compounds IIIa-n, IVa-n and Va-n. Alternatively the corresponding aminonitrile, cyanohydrin or a mixture of the corresponding aldehyde, hydrocyanic acid and ammonia or also a mixture of the corresponding aldehyde, ammonium and cyanide salts can also be used as hydantoin precursors.

Another preferred embodiment of the method according to the invention comprises the following steps:

a) Reaction of the urea derivative according to formulae IIIa-n, IVa-n or Va-n with the amino acid to a diketopiperazine VIa-m of formula,

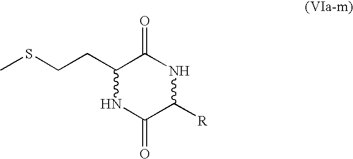

with R as previously defined;
b) Reaction of the diketopiperazine VI to a mixture of dipeptides with the formulae DD/LL/DL/LD-I and DD/LL/DL/LD-II:

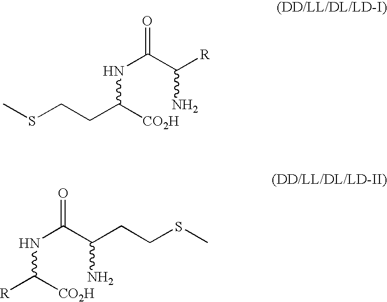

with R as previously defined.

Reaction of the urea derivative according to formulae IIIn, IVn and Vn to a diketopiperazine VIa-m and the further reaction of the diketopiperazine to a diastereomeric mixture with the preferred dipeptides L-EAA-DL-methionine (I) and DL-methionyl-L-EAA (II) is shown in Scheme 4:

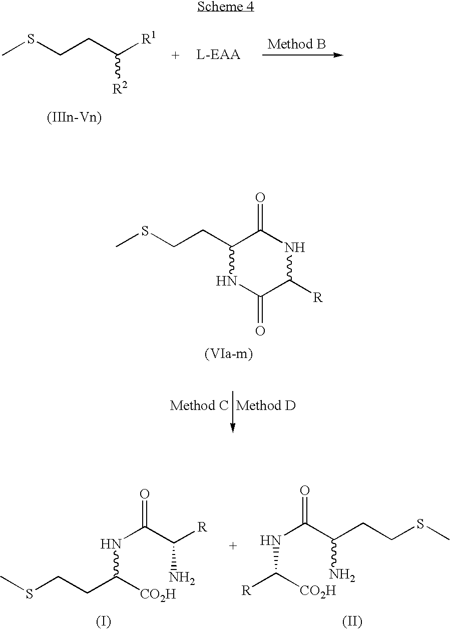

The reaction of the diketopiperazine VIa-m to a mixture of the preferred dipeptides L-EAA-DL-methionine (I) and DL-methionyl-L-EAA (II). This method comprises the methods B, C and D presented in Scheme 4. In these methods, in each case diketopiperazine VIa-m is formed as an intermediate.

The reaction of the urea derivative with the amino acid to the diketopiperazine is preferably carried out at a temperature from 20° C. to 200° C., preferably from 40° C. to 180° C. and especially preferably from 100° C. to 170° C.

In a preferred method, the reaction of the urea derivative with the amino acid to the diketopiperazine takes place under pressure, preferably at a pressure from 2 to 90 bar, especially preferably at a pressure from 4 to 70 bar, quite especially preferably at a pressure from 5 to 50 bar.

The reaction of the urea derivative with the amino acid to the diketopiperazine preferably takes place in the presence of a base. The base is preferably selected from the group comprising nitrogen-containing bases, NH₄HCO₃, (NH₂CO₃, KHCO₃, K₂CO₃, NH₄OH/CO₂ mixture, carbamate salts, alkali and alkaline-earth bases.

In another preferred method the reaction to the diketopiperazine either takes place by reaction of the urea derivative of formula,

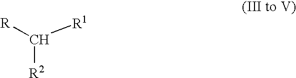

with R denoting a methionyl residue, with an amino acid, selected from the group comprising lysine, threonine, tryptophan, histidine, valine, leucine, isoleucine, phenylalanine, arginine, cysteine or cystine
or
by reaction of the urea derivative of formula,

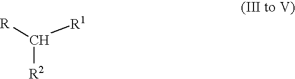

where R is an amino acid residue selected from the group comprising lysine, threonine, tryptophan, histidine, valine, leucine, isoleucine, phenylalanine, arginine, cysteine or cystine, with the amino acid methionine.

In the preferred method in which the reaction of the urea derivative to the diketopiperazine takes place by reaction with methionine, a ratio of urea derivative to methionine from 1:100 to 1:0.5 is especially preferred.

In another preferred method the reaction of the diketopiperazine to a mixture of dipeptides of formula I and II takes place by acid hydrolysis. Preferably the reaction of the diketopiperazine to a mixture of L-EAA-DL-methionine (I) and DL-methionyl-L-EAA (II) takes place by acid hydrolysis.

The acid hydrolysis is carried out in the presence of an acid, which is preferably selected from the group comprising the mineral acids, HCl, H₂CO₃, CO₂/H₂O, H₂SO₄, phosphoric acids, carboxylic acids and hydroxycarboxylic acids.

In another embodiment of the method according to the invention the reaction of the diketopiperazine to a mixture of dipeptides of formula (I) and (II) takes place by basic hydrolysis. Preferably the reaction of the diketopiperazine to a mixture of L-EAA-DL-methionine (I) and DL-methionyl-L-EAA (II) takes place by basic hydrolysis.

Basic hydrolysis is preferably carried out at a pH from 7 to 14, especially preferably at a pH from 8 to 13, quite especially preferably at a pH from 9 to 12. Complete racemization may occur. Basic conditions can be provided by using a substance that is preferably selected from the group comprising nitrogen-containing bases, NH₄HCO₃, (NH₄)₂CO₃, NH₄OH/CO₂ mixture, carbamate salts, KHCO₃, K₂CO₃, carbonates, alkali and alkaline-earth bases.

The acid or basic hydrolysis is preferably carried out at temperatures from 50° C. to 200° C., preferably from 80° C. to 180° C. and especially preferably from 90° C. to 160° C.

In a preferred method the amino acid residue of the urea derivative III to V is in the D- or L-configuration or in a mixture of D- and L-configuration, preferably in a mixture of D- and L-configuration, if the urea derivative is derived from methionine.

In another preferred method the amino acid residue of the urea derivative III to V is in the D- or L-configuration or in a mixture of D- and L-configuration, preferably in the L-configuration, if the urea derivative is derived from an amino acid selected from the group comprising lysine, threonine, tryptophan, histidine, valine, leucine, isoleucine, phenylalanine, arginine, cysteine, cystine.

In another preferred method, dipeptides are obtained as a mixture of LL, DL, LD and DD, preferably as a mixture of LL, LD, DL.

In a preferred method the diketopiperazine is isolated before the hydrolysis. It is preferable for the diketopiperazine to be isolated by crystallization from the reaction solution, preferably at a temperature from −30 to 120° C., especially preferably at a temperature from 10 to 70° C.

For isolation of the diastereomeric mixture of the dipeptides of formula DD/LL/DL/LD-(I) and DD/LL/DL/LD-(II), preferably of the diastereomeric mixture of L-EAA-DL-methionine (I) and DL-methionyl-L-EAA (II), from basic reaction solutions, it is acidified and obtained by crystallization or precipitation. A pH value from 2 to 10 is preferred, a pH value from 3 to 9 is especially preferred, and the corresponding isoelectric point of the respective dipeptide of formula I and II is quite especially preferred. Acids preferably from the group comprising the mineral acids, HCl, H₂CO₃, CO₂/H₂O, H₂SO₄, phosphoric acids, carboxylic acids and hydroxycarboxylic acids can be used for the acidification.

For isolation of the diastereomeric mixture of the dipeptides of formula DD/LL/DL/LD-(I) and DD/LL/DL/LD-(II), preferably of the diastereomeric mixture of L-EAA-DL-methionine (I) and DL-methionyl-L-EAA (II), from acidic reaction solutions, after neutralization by adding bases it is obtained by crystallization or precipitation. A pH value from 2 to 10 is preferred, a pH value from 3 to 9 is especially preferred, and the corresponding isoelectric point of the respective dipeptide of formula I and II is quite especially preferred. The bases used for neutralization are preferably from the group comprising NH₄HCO₃, (NH₄)₂CO₃, nitrogen-containing bases, NH₄OH, carbamate salts, KHCO₃, K₂CO₃, carbonates, alkali and alkaline-earth bases.

Another alternative embodiment of the method according to the invention comprises the synthesis of the non-natural dipeptides L-EAA-D-methionine Ia-Ij or D-methionyl-L-EAA IIa-IIj using protecting group technology. Thus, for synthesis of the dipeptides L-EAA-D-methionine (LD-I) the amino group of the free L-EAA was first protected with the BOC protecting group (tert-butoxycarbonyl-). Alternatively, the Z protecting group (benzoxycarbonyl-) could also be used successfully. D-methionine was esterified with methanol, so that the acid function was protected. Then the coupling reaction of the BOC- or Z-protected L-EAA with D-methionine methyl ester was carried out using DCC (dicyclohexylcarbodiimide) (see Scheme 5).

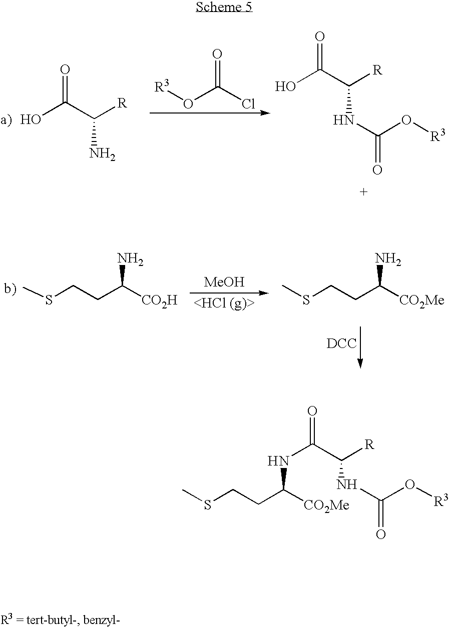

After purification of BOC-L-EAA-D-methionine-OMe or Z-L-EAA-D-methionine-OMe, first the methyl ester was cleaved under mild, basic conditions. Finally the BOC or Z protecting group was cleaved acidically with HBr in glacial acetic acid and the free dipeptide L-EAA-D-methionine (LD-I) was purified by reprecipitation and recrystallization (see Scheme 6).

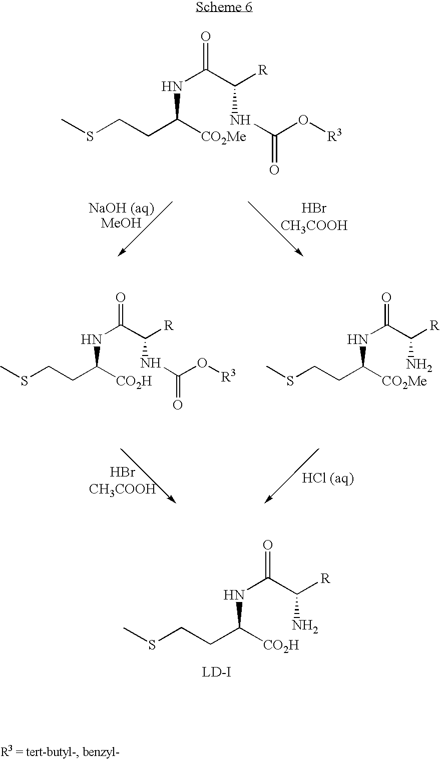

Alternatively the BOC-protected dipeptide methyl ester BOC-L-EAA-D-methionine-OMe could also first be reacted with HBr in glacial acetic acid, thus removing the BOC protecting group. After concentration by evaporation, the methyl ester could then be cleaved by adding dilute hydrochloric acid solution. The free dipeptide L-EAA-D-methionine (LD-I) could once again be purified by reprecipitation and recrystallization (see Scheme 6).

It was also possible to transfer the complete route for the dipeptides L-EAA-D-methionine Ia-Ij. In this case the methyl esters of L-EAA and BOC- or Z-protected D-methionine were used.

All the stated methods of the present invention are preferably carried out in an aqueous medium.

Furthermore, the methods of the present invention can be carried out in batch methods or in continuous methods, which are known by a person skilled in the art

### EXAMPLES

### Example 1

### General Method for the Synthesis of the Non-Natural Dipeptides L-EAA-D-Methionine Ia-Ij or D-Methionyl-L-EAA IIa-Iij Using Protecting Group Technology

For synthesis of the dipeptides L-EAA-D-methionine (LD-I), the amino group of the free L-EAA was first protected with the BOC protecting group (tert-butoxycarbonyl-). Alternatively, the Z protecting group (benzoxycarbonyl-) could also be used successfully. D-methionine was esterified with methanol, so that the acid function was protected. Then the coupling reaction of the BOC- or Z-protected L-EAA with D-methionine methyl ester was carried out using DCC (dicyclohexylcarbodiimide) (see Scheme 5).

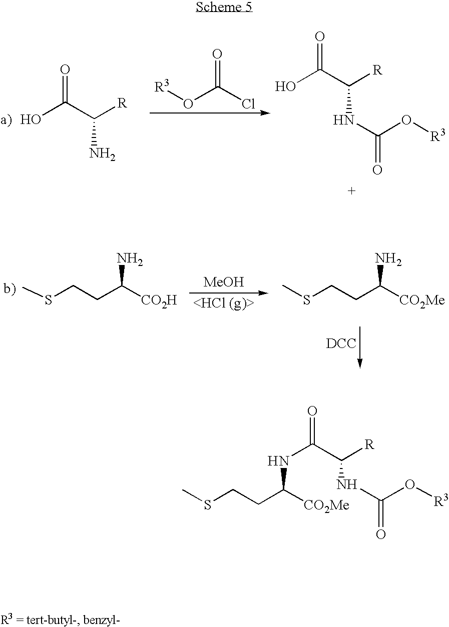

After purification of BOC-L-EAA-D-methionine-OMe or Z-L-EAA-D-methionine-OMe first the methyl ester was cleaved under mild, basic conditions. Finally the BOC or Z protecting group was cleaved acidically with HBr in glacial acetic acid and the free dipeptide L-EAA-D-methionine (LD-I) was purified by reprecipitation and recrystallization (see Scheme 6).

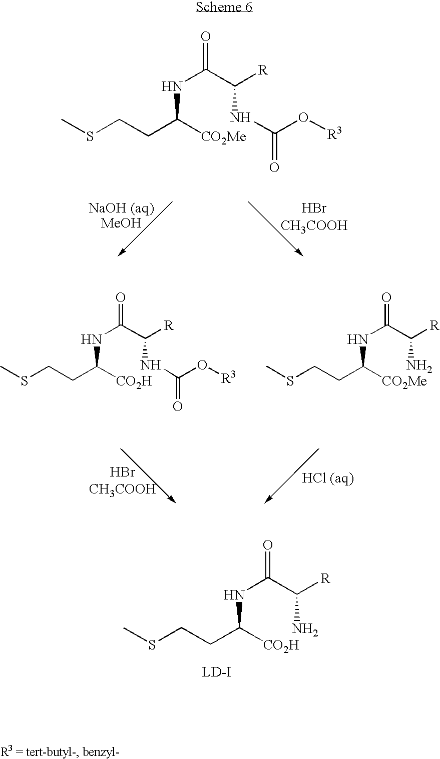

Alternatively the BOC-protected dipeptide methyl ester BOC-L-EAA-D-methionine-OMe could also be reacted first with HBr in glacial acetic acid, thus removing the BOC protecting group. After concentration by evaporation, the methyl ester could then be cleaved by adding dilute hydrochloric acid solution. The free dipeptide L-EAA-D-methionine (LD-I) could then once again be purified by reprecipitation and recrystallization (see Scheme 6).

It was also possible to transfer the complete route for the dipeptides L-EAA-D-methionine Ia-Ij. For this, the methyl esters of L-EAA and BOC- or Z-protected D-methionine were used.

### Example 2

### a) Specification for Synthesis of Z-D-Met

30.0 g (0.201 mol) of D-methionine and 42.4 g (0.4 mol) of Na₂CO₃ were put in 200 ml of water and cooled to 0° C. on an ice bath. Then 51.2 g (0.3 mol) of carboxybenzyloxychloride (Cbz-Cl) was added slowly and the reaction mixture was stirred for 3 hours at room temperature. Then it was acidified with dilute hydrochloric acid and the reaction solution was extracted three times with 50 ml MTBE each time. The combined organic phases were dried over MgSO₄ and concentrated in the rotary evaporator. The residue obtained was recrystallized from diethyl ether/ethyl acetate and dried under vacuum at 30° C. 36.4 g (64% of carboxybenzyloxy-D-methionine (Z-D-Met) was isolated as a white crystalline solid.

### b) General Specification for Synthesis of Z-L-EAA

50 mmol L-EAA and 10.6 g (100 mmol) of Na₂CO₃ were put in 50 ml of water and cooled to 0° C. on an ice bath. Then 12.8 g (75 mmol) of carboxybenzyloxychloride (Cbz-Cl) was added slowly and the reaction mixture was stirred for 3 hours at room temperature. Then it was acidified with dilute hydrochloric acid and the reaction solution was extracted three times with 25 ml MTBE each time. The combined organic phases were dried over MgSO₄ and concentrated in the rotary evaporator. The residue obtained was recrystallized and dried under vacuum at 30° C.

### Example 3

### Specification for Synthesis of D-Met-OMe×HCl

50.0 g (0.335 mol) of D-methionine was suspended in 500 ml methanol and HCl gas was passed through at a moderate rate until saturated. The methionine dissolved and the solution heated up to 55° C. Then the reaction mixture was stirred overnight at room temperature. Next morning, the mixture was concentrated to dryness in the rotary evaporator at 40° C. and the residue obtained was recrystallized twice from diethyl ether. 47.1 g (86%) of D-methionine methyl ester hydrochloride was isolated as a white crystalline solid.

### Example 4

### General Specification for Synthesis of L-EAA-OMe×HCl

0.3 mol L-EAA was suspended in 500 ml methanol and HCl gas was passed through at a moderate rate until saturated. The amino acid dissolved and the solution heated up to 50-60° C. The reaction mixture was stirred overnight at room temperature. Next morning, the mixture was concentrated to dryness in the rotary evaporator at 40° C. and the residue obtained was recrystallized twice from diethyl ether or diethyl ether/methanol mixture.

### Example 5

### General Specification for Synthesis of Compounds of the Group PG-D-Met-L-EAA-OMe (PG-DL-II-OMe) (Coupling Reaction)

20.0 mmol L-EAA-OMe hydrochloride was suspended in a mixture of 30 ml chloroform and 5 ml methanol, 4.15 g (30 mmol) of K₂CO₃ was added and it was stirred for 1 hour at room temperature. Then the salt was filtered off and washed with a little chloroform. After concentration of the filtrate by evaporation, the residue obtained was taken up in 50 ml tetrahydrofuran, 4.37 g (21.0 mmol; 1.05 eq.) DCC and 5.66 g (20.0 mmol) of Z-D-methionine were added and it was stirred for 16 h at room temperature. Then 3 ml glacial acetic acid was added to the reaction mixture, stirred for 30 minutes and the precipitated white solid (N,N′-dicyclohexylurea) was filtered off. The filtrate was concentrated in the rotary evaporator and any precipitated N,N′-dicyclohexylurea was filtered off. The oily residue was then recrystallized twice from chloroform/n-hexane and dried under oil-pump vacuum.

PG: protecting group (Z or BOC protecting group)

5a) Z-D-Met-L-Val-OMe (Z-DL-IIa-OMe)

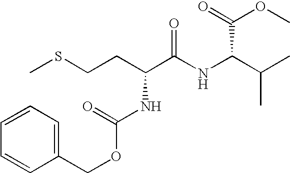

Empirical formula: C₁₉H₂₈N₂O₅S (396.50 g/mol), yield: 4.60 g (58%), purity: 97%, white solid.

¹H-NMR of Z-D-Met-L-Val-OMe (Z-DL-IIa-OMe) (500 MHz, CDCl₃): δ=0.88 (d, ³J=6.8 Hz, 3H, CH₃); 0.93 (d, ³J=6.8 Hz, 3H, CH₃); 1.90-2.20 (m, 3H, SCH₂CH₂, CH(CH₃)₂); 2.10 (s, 3H, SCH₃); 2.50-2.64 (m, 2H, SCH₂); 3.73 (s, 3H, OCH₃); 4.38-4.44 (m, 1H, CH); 4.48-4.54 (m, 1H, CH); 5.08-5.18 (m, 2H, OCH₂); 5.49 (bs, 1H, NH); 6.58 (bs, 1H, NH); 7.24-7.38 (m, 5H, Ph)

¹³C-NMR of Z-D-Met-L-Val-OMe (Z-DL-IIa-OMe) (125 MHz, CDCl₃): δ=15.26; 17.74; 19.01; 30.13; 31.16; 31.67; 52.21; 57.24; 67.22; 128.16; 128.27; 128.58; 136.16; 156.13; 171.01; 171.95

5b) Z-D-Met-L-Leu-OMe (Z-DL-IIb-OMe)

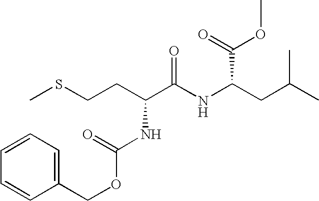

Empirical formula: C₂₀H₃₀N₂O₅S (410.53 g/mol), yield: 5.40 g (66%), purity: 97%, white solid.

¹H-NMR of Z-D-Met-L-Leu-OMe (Z-DL-IIb-OMe) (500 MHz, d₆-DMSO): δ=0.90-0.95 (m, 6H, CH(CH₃)₂); 1.50-1.72 (m, 3H, CH₂CH(CH₃)₂); 1.90-2.15 (m, 2H, SCH₂CH₂); 2.09 (s, 3H, SCH₃); 2.48-2.64 (m, 2H, SCH₂); 3.71 (s, 3H, OCH₃); 4.36-4.44 (m, 1H, CH); 4.56-4.62 (m, 1H, CH); 5.12 (s, 2H, OCH₂); 5.56 (d, ³J=7.6 Hz, 1H, OC(═O)NH); 6.59 (bs, 1H, NH); 7.26-7.36 (m, 5H, Ph)

¹³C-NMR of Z-D-Met-L-Leu-OMe (Z-DL-IIb-OMe) (125 MHz, d₆-DMSO): δ=15.27; 21.86; 22.78; 24.95; 30.11; 31.62; 33.96; 41.35; 50.86; 52.33; 67.20; 128.09; 128.25; 128.57; 156.97; 170.95; 173.01

5c) Z-D-Met-L-Ile-OMe (Z-DL-IIc-OMe)

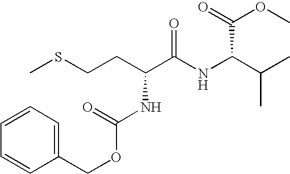

Empirical formula: C₂₀H₃₀N₂O₅S (410.53 g/mol), yield: 5.09 g (62%), purity: 97%, white solid.

¹H-NMR of Z-D-Met-L-Ile-OMe (Z-DL-IIc-OMe) (500 MHz, CDCl₃): δ=0.86-0.94 (m, 6H, CH(CH₃)CH₂CH₃); 1.10-1.45 (m, 2H, CH₂CH₃); 1.84-1.94 (m, 1H, CH(CH₃); 1.94-2.16 (m, 2H, SCH₂CH₂); 2.10 (s, 3H, SCH₃); 2.49-2.64 (m, 2H, SCH₂); 3.72 (s, 3H, OCH₃); 4.36-4.44 (m, 1H, CH); 4.52-4.58 (m, 1H, CH); 5.08-5.18 (m, 2H, OCH₂); 5.46 (bs, 1H, NH); 6.58 (bs, 1H, NH); 7.28-7.38 (m, 5H, Ph)

¹³C-NMR of Z-D-Met-L-Ile-OMe (Z-DL-IIc-OMe) (125 MHz, CDCl₃): δ=11.55; 15.26; 15.54; 25.19; 30.12; 31.70; 33.96; 37.79; 52.15; 45.07; 56.55; 67.18; 128.12; 128.24; 128.56; 156.13; 170.92; 171.96

5d) Z-D-Met-L-Thr-OMe (Z-DL-IId-OMe)

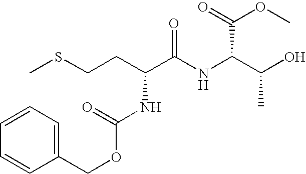

Empirical formula: C₁₈H₂₆N₂O₆S (398.47 g/mol), yield: 2.14 g (36%), purity: 95%, slightly yellowish solid.

¹H-NMR of Z-D-Met-L-Thr-OMe (Z-DL-IId-OMe) (500 MHz, CDCl₃): δ=1.10-1.25 (m, 3H, CHCH₃); 1.95-2.20 (m, 2H, SCH₂CH₂); 2.09 (s, 3H, SCH₃); 2.49 (bs, 1H, OH); 2.52-2.62 (m, 2H, SCH₂); 3.74 (s, 3H, OCH₃); 4.30-4.56 (m, 3H, 3×CH); 5.12 (s, 2H, OCH₂); 5.70-5.78 (m, 1H, NH); 7.03 (d, ³J=8.9 Hz, 1H, NH); 7.28-7.38 (m, 5H, Ph)

¹³C-NMR of Z-D-Met-L-Thr-OMe (Z-DL-IId-OMe) (125 MHz, CDCl₃): δ=15.15; 20.05; 30.10; 31.91; 52.66; 54.37; 57.44; 67.23; 67.82; 128.17; 128.26; 128.57; 136.16; 156.18; 171.25; 171.87

5e) Z-D-Met-L-Lys (BOC)—OMe (Z-DL-IIe(BOC)—OMe)

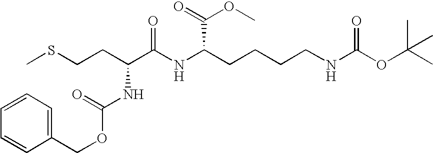

Empirical formula: C₂₅H₃₉N₃O₇S (525.66 g/mol), yield: 10.86 g (33%), purity: 95%, slightly yellowish solid.

¹H-NMR of Z-D-Met-L-Lys(BOC)—OMe (Z-DL-IIe(BOC)—OMe) (500 MHz, CDCl₃): δ=1.25-1.90 (m, 6H, 3×CH₂(Lys)); 1.43 (s, 9H, C(CH₃)₃); 1.92-2.16 (m, 2H, SCH₂CH₂); 2.09 (s, 3H, SCH₃); 2.48-2.62 (m, 2H, SCH₂); 3.02-3.12 (m, 2H, NCH₂); 3.72 (s, 3H, OCH₃); 4.35-4.65 (m, 3H, 2×CH, NH); 5.13 (s, 2H, OCH₂); 5.58 (d, ³J=7.5 Hz, 1H, NH); 6.75 (bs, 1H, NH); 7.28-7.36 (m, 5H, Ph)

¹³C-NMR of Z-D-Met-L-Lys(BOC)—OMe (Z-DL-IIe(BOC)—OMe) (125 MHz, CDCl₃): δ=15.31; 22.44; 28.45; 29.47; 30.12; 31.82; 52.08; 52.45; 67.20; 79.15; 128.08; 128.25; 128.34; 128.57; 156.07; 170.97; 172.38

5f) Z-D-Met-L-Phe-OMe (Z-DL-IIg-OMe)

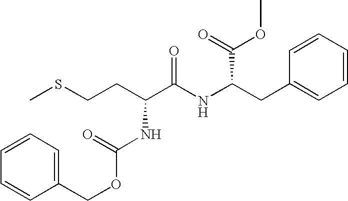

Empirical formula: C₂₃H₂₈N₂O₅S (444.54 g/mol), yield: 3.73 g (42%), purity: 95% (HPLC), white solid.

¹H-NMR of Z-D-Met-L-Phe-OMe (Z-DL-IIg-OMe) (500 MHz, d₆-DMSO/CDCl₃): δ=1.72-1.94 (m, 2H, SCH₂CH₂); 2.01 (s, 3H, SCH₃); 2.30-2.38 (m, 2H, SCH₂); 2.94-3.14 (m, 2H, CH₂Ph); 3.70 (s, 3H, OCH₃); 4.25-4.32 (m, 1H, CHCH₂CH₂S); 4.70-4.78 (m, 1H, CHCH₂Ph); 5.00-5.10 (bs, 2H, OCH₂Ph); 6.60-6.70 (m, 1H, NH); 7.10-7.35 (m, 10H, 2×Ph); 7.75-7.80 (bs, 1H, NH)

5g) Z-D-Met-L-His-OMe (Z-DL-IIh-OMe)

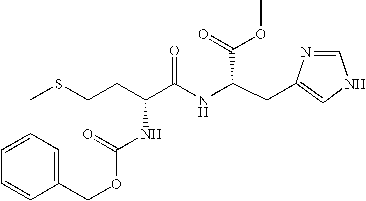

Empirical formula: C₂₀H₂₆N₄O₅S (434.51 g/mol), yield: 2.35 g (27%), purity: 95% (HPLC), slightly yellowish solid.

¹H-NMR of Z-D-Met-L-His-OMe (Z-DL-IIh-OMe) (500 MHz, CDCl₃): δ=1.88-2.14 (m, 2H, SCH₂CH₂); 2.05 (s, 3H, SCH₃); 2.44-2.56 (m, 2H, SCH₂); 3.06-3.14 (m, 2H, CH₂-imidazolyl); 3.68 (s, 3H, OCH₃); 4.20-4.40 (m, 2H, NH, CH); 4.70-4.76 (m, 1H, CH); 5.11 (s, 2H, OCH₂); 5.91 (d, ³J=7.6 Hz, 1H, NH); 6.76 (bs, 1H, CH(imidazolyl); 7.26-7.45 (m, 5H, Ph); 7.73 (bs, 1H, CH(imidazolyl)); 9.30 (bs, 1H, NH)

¹³C-NMR of Z-D-Met-L-His-OMe (Z-DL-IIh-OMe) (125 MHz, CDCl₃): δ=15.27; 29.94; 31.81; 33.92; 52.46; 67.14; 116.88; 128.02; 128.12; 128.23; 128.49; 128.58; 133.23; 135.20; 136.21; 156.97; 171.17; 171.57

5h) Z-D-Met-L-Trp-OMe (Z-DL-IIj-OMe)

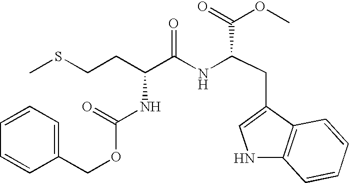

Empirical formula: C₂₅H₂₉N₃O₅S (483.58 g/mol), yield: 5.71 g (59%), purity: 98% (HPLC), slightly yellowish solid.

¹H-NMR of Z-D-Met-L-Trp-OMe (Z-DL-IIj-OMe) (500 MHz, d₆-DMSO): δ=1.60-1.80 (m, 2H, SCH₂CH₂); 1.95 (s, 1H, SCH₃); 2.25-2.35 (m, 2H, SCH₂); 3.02-3.20 (m, 2H, CH₂-indolyl); 3.60 (s, 3H, OCH₃); 4.10-4.16 (m, 1H, CH); 4.50-4.60 (m, 1H, CH); 4.98-5.08 (m, 2H, OCH₂); 6.94-7.50 (m, 12H, indolyl, Ph, OC(═O)NH); 8.25 (d, ³J=8.6 Hz, 1H, CONH-Trp)

¹³C-NMR of Z-D-Met-L-Trp-OMe (Z-DL-IIj-OMe) (125 MHz, d₆-DMSO): δ=14.42; 27.01; 29.40; 31.59; 51.75; 52.78; 53.60; 65.36; 109.16; 111.31; 117.84; 118.31; 120.86; 123.60; 126.90; 127.59; 127.68; 128.21; 136.02; 136.89; 155.81; 171.32; 172.06

### Example 6

### General Specification for Synthesis of Compounds of the Group PG-L-EAA-D-Met-OMe (PG-LD-1-OMe) (Coupling Reaction)

3.99 g (20.0 mmol) of D-methionine methyl ester hydrochloride was suspended in a mixture of 30 ml chloroform and 5 ml methanol, 4.15 g (30 mmol) of K₂CO₃ was added and it was stirred for 1 hour at room temperature. Then the salt was filtered off and washed with a little chloroform. After concentration of the filtrate by evaporation, the residue obtained was taken up in 50 ml tetrahydrofuran, 4.37 g (21.0 mmol; 1.05 eq.) DCC and 20.0 mmol of the corresponding PG-L-EAA (PG-L-amino acid) were added and it was stirred for 16 h at room temperature. Then 3 ml of glacial acetic acid was added to the reaction mixture, it was stirred for 30 minutes and the precipitated white solid (N,N′-dicyclohexylurea) was filtered off. The filtrate was concentrated in the rotary evaporator and any precipitated N,N′-dicyclohexylurea was filtered off. The oily residue was then recrystallized twice from chloroform/n-hexane and dried under oil-pump vacuum.

PG: protecting group (Z or BOC protecting group)

6a) Z-L-Val-D-Met-OMe (Z-LD-Ia-OMe)

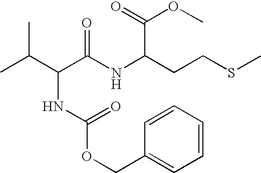

Empirical formula: C₁₉H₂₈N₂O₅S (396.50 g/mol), yield: 3.01 g (38%), purity: 95% (HPLC), white solid

¹H-NMR of Z-L-Val-D-Met-OMe (Z-LD-Ia-OMe) (500 MHz, CDCl₃): δ=0.92 (d, ³J=6.9 Hz, 3H, CH₃); 0.99 (d, ³J=6.9 Hz, 3H, CH₃); 1.90-2.25 (m, 3H, SCH₂CH₂, CH(CH₃)₂); 2.07 (s, 3H, SCH₃); 2.44-2.54 (m, 2H, SCH₂); 3.74 (s, 3H, OCH₃); 4.04-4.10 (m, 1H, CH); 4.67-4.74 (m, 1H, CH); 5.12 (s, 2H, OCH₂); 5.28 (bs, 1H, NH); 6.65 (d, ³J=7.5 Hz, 1H, NH); 7.28-7.38 (m, 5H, Ph)

¹³C-NMR of Z-L-Val-D-Met-OMe (Z-LD-Ia-OMe) (125 MHz, CDCl₃): δ=15.45; 17.46; 19.30; 29.96; 30.87; 31.40; 51.57; 52.55; 60.37; 67.18; 128.08; 128.24; 128.57; 136.19; 156.38; 171.04; 172.04

6b) Z-L-Leu-D-Met-OMe (Z-LD-Ib-OMe)

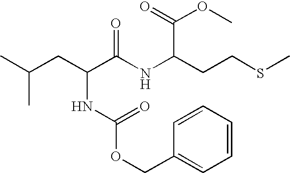

Empirical formula: C₂₀H₃₀N₂O₅S (410.53 g/mol), yield: 4.48 g (55%), purity: 96% (HPLC), white solid

¹H-NMR of Z-L-Leu-D-Met-OMe (Z-LD-Ib-OMe) (500 MHz, CDCl₃): δ=0.94 (d, ³J=6.3 Hz, 6H, CH(CH₃)₂); 1.48-1.72 (m, 3H, CH₂CH(CH₃)₂); 1.90-2.20 (m, 2H, SCH₂CH₂); 2.07 (s, 3h, SCH₃); 2.42-2.52 (m, 2H, SCH₂); 3.73 (s, 3H, OCH₃); 4.20-4.30 (m, 1H, CH); 4.64-4.72 (m, 1H, CH); 5.12 (s, 2H, OCH₂); 5.23 (d, ³J=7.9 Hz, 1H, NH); 6.84 (d, ³J=7.2 Hz, 1H, NH); 7.28-7.38 (m, 5H, Ph)

¹³C-NMR of Z-L-Leu-D-Met-OMe (Z-LD-Ib-OMe) (125 MHz, CDCl₃): δ=15.47; 22.97; 24.81; 29.97; 31.46; 51.58; 52.55; 67.23; 128.09; 128.26; 128.58; 136.16; 156.23; 172.02; 172.09

6c) Z-L-Ile-D-Met-OMe (Z-LD-Ic-OMe)

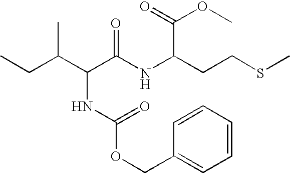

Empirical formula: C₂₀H₃₀N₂O₅S (410.53 g/mol), yield: 3.89 g (47%), purity: 97% (HPLC), white solid

¹H-NMR of Z-L-Ile-D-Met-OMe (Z-LD-Ic-OMe) (500 MHz, CDCl₃): δ=0.91 (t, ³J=7.1 Hz, 3H, CH₂CH₃); 0.96 (d, ³J=7.1 Hz; 3H, CH(CH₃); 1.08-1.16 (m, 1H, CH′H″CH₃); 1.46-1.54 (m, 1H, CH′H″CH₃); 1.88-2.20 (m, 3H, CH(CH₃), SCH₂CH₂); 2.07 (s, 3H, SCH₃); 2.44-2.52 (m, 2H, SCH₂); 3.73 (s, 3H, OCH₃); 4.08-4.16 (m, 1H, CH); 4.66-4.74 (m, 1H, CH); 5.11 (s, 2H, OCH₂); 5.34 (d, ³J=7.6 Hz, 1H; NH); 6.74 (d, ³J=8.0 Hz, 1H, NH); 7.28-7.38 (m, 5H, Ph)

¹³C-NMR of Z-L-Ile-D-Met-OMe (Z-LD-Ic-OMe) (125 MHz, CDCl₃): δ=11.54; 15.46; 15.68; 24.66; 29.96; 31.42; 37.36; 51.59; 52.57; 59.83; 67.19; 128.10; 128.25; 128.58; 136.20; 156.34; 170.99; 172.03

6d) Z-L-Thr-D-Met-OMe (Z-LD-Id-OMe)

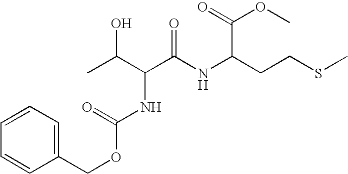

Empirical formula: C₁₈H₂₆N₂O₆S (398.47 g/mol), yield: 2.47 g (31%), purity: 99% (HPLC), slightly yellowish solid

¹H-NMR of Z-L-Thr-D-Met-OMe (Z-LD-Id-OMe) (500 MHz, CDCl₃): δ=1.19 (d, ³J=6.4 Hz, 3H, CH₃); 1.94-2.20 (m, 2H, SCH₂CH₂); 2.06 (s, 3H, SCH₃); 2.45-2.55 (m, 2H, SCH₂); 3.73 (s, 3H, OCH₃); 4.18 (bs, 1H, CH); 4.39 (bs, 1H; CH); 4.66-4.74 (m, 1H, CH); 5.10-5.18 (m, 2H, OCH₂); 5.85 (bs, 1H, OC(═O)NH); 7.21 (bs, 1H, NH); 7.28-7.38 (m, 5H, Ph)

¹³C-NMR of Z-L-Thr-D-Met-OMe (Z-LD-Id-OMe) (125 MHz, CDCl₃): δ=15.43; 18.48; 30.10; 30.91; 51.80; 52.66; 59.16; 66.99; 67.36; 128.04; 128.29; 128.59; 136.08; 156.94; 171.27; 172.25

6e) BOC-L-Lys(BOC)-D-Met-OMe (BOC-LD-Ie(BOC)—OMe)

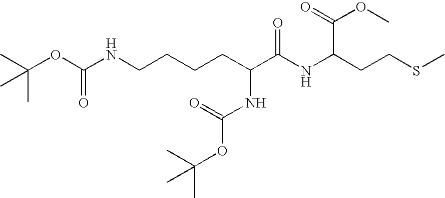

Empirical formula: C₂₂H₄₁N₃O₇S (491.64 g/mol), yield: 5.22 g (53.1%), purity: 97% (HPLC), white amorphous solid

¹H-NMR of BOC-L-Lys(BOC)-D-Met-OMe (BOC-LD-Ie(BOC)—OMe) (500 MHz, CDCl₃): δ=1.32-1.42 (m, 2H, CH₂(Lys)); 1.44 (s, 9H, C(CH₃)₃); 1.45 (s, 9H, C(CH₃)₃); 1.46-1.56 (m, 2H, CH₂(Lys)); 1.60-1.72 (m, 1H, CHCH′H″(Lys)); 1.82-1.92 (m, 1H, CHCH′CH″(Lys); 1.92-2.03 (m, 1H, SCH₂CHH′H″); 2.09 (s, 3H, SCH₃); 2.12-2.22 (m, 1H, SCH₂CH′H″); 2.51 (t, ³J=7.4 Hz, 2H, SCH₂); 3.08-3.16 (m, 2H, NCH₂); 3.75 (s, 3H, OCH₃); 4.02-4.12 (m, 1H, CH); 4.54-4.62 (m, 1H, NH); 4.66-4.74 (m, 1H, CH): 5.06-4.14 (m, 1H, NH); 6.81 (d, ³J=7.4 Hz, 1H, NH)

6f) Z-L-Phe-D-Met-OMe (Z-LD-Ig-OMe)

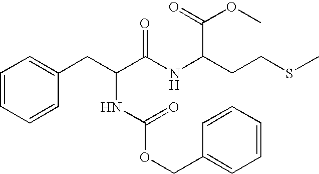

Empirical formula: C₂₃H₂₈N₂O₅S (444.54 g/mol), yield: 3.51 g (40%), purity: 99% (HPLC), white solid

¹H-NMR of Z-L-Phe-D-Met-OMe (Z-LD-Ig-OMe) (500 MHz, CDCl₃): δ=1.78-2.04 (m, 2H, SCH₂CH₂); 2.02 (s, 3H, SCH₃); 2.20-2.30 (m, 2H, SCH₂); 3.02-3.14 (m, 2H, CH₂Ph); 3.71 (s, 3H, OCH₃); 4.40-4.50 (m, 1H, CH); 4.60-4.66 (m, 1H, CH); 5.09 (s, 2H, OCH₂); 5.31 (bs, 1H, OC(═O)NH); 6.42 (d, ³J=7.6 Hz, 1H, NH); 7.16-7.36 (m, 10H, 2×Ph)

¹³C-NMR of Z-L-Phe-D-Met-OMe (Z-LD-Ig-OMe) (125 MHz, CDCl₃): δ=15.37; 29.67; 31.35; 38.63; 51.52; 52.53; 56.36; 67.15; 127.18; 128.06; 128.24; 128.57; 128.83; 129.26; 136.13; 136.30; 155.90; 170.63; 171.88

6g) BOC-L-Phe-D-Met-OMe (BOC-LD-Ig-OMe)

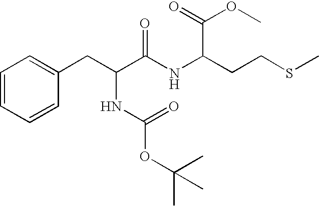

Empirical formula: C₂₀H₃₀N₂O₅S (410.53 g/mol), yield: 4.03 g (49%), purity: 98% (HPLC), white solid

¹H-NMR of BOC-L-Phe-D-Met-OMe (BOC-LD-Ig-OMe) (500 MHz, CDCl₃): δ=1.42 (s, 9H, C(CH₃)₃); 1.80-2.08 (m, 2H, SCH₂CH₂); 2.04 (s, 3H, SCH₃); 2.24-2.34 (m, 2H, SCH₂); 3.07 (d, ³J=7.2 Hz, 2H, CH₂Ph); 3.73 (s, 3H, OCH₃); 4.30-4.42 (m, 1H, CH); 4.60-4.68 (m, 1H, CH); 4.90-5.02 (bs, 1H, NH); 6.44 (d, ³J=7.9 Hz, 1H, NH); 7.18-7.34 (m, 5H, Ph)

¹³C-NMR of BOC-L-Phe-D-Met-OMe (BOC-LD-Ig-OMe) (125 MHz, CDCl₃): δ=15.39; 28.29; 29.67; 31.51; 38.42; 51.47; 52.50; 56.00; 80.38; 127.07; 128.79; 129.27; 136.60; 156.42; 171.00; 171.94

6h) Z-L-His-D-Met-OMe (Z-LD-Ih-OMe)

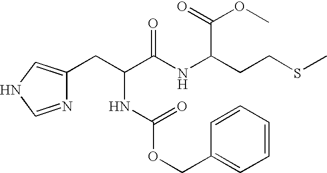

Empirical formula: C₂₀H₂₆N₄O₅S (434.51 g/mol), yield: 1.65 g (19%), purity: 95% (HPLC), slightly yellowish solid

¹H-NMR of Z-L-His-D-Met-OMe (Z-LD-Ih-OMe) (500 MHz, d₆-DMSO/CDCl₃): δ=1.82-1.98 (m, 2H, SCH₂CH₂); 2.01 (s, 3H, SCH₃); 2.30-2.44 (m, 2H, SCH₂); 2.76-2.94 (m, 2H, CH₂-imidazolyl); 3.63 (s, 3H, OCH₃); 4.28-4.42 (m, 2H, 2×CH); 5.01 (s, 2H, OCH₂); 6.78 (bs, 1H, CH(imidazolyl)); 7.25-7.37 (m, 6H, Ph, NH); 7.50 (bs, 1H, CH(imidazolyl)); 8.27 (bs, 1H, NH); 11.76 (bs, 1H, NH(imidazolyl))

¹³C-NMR of Z-L-His-D-Met-OMe (Z-LD-Ih-OMe) (125 MHz, d₆-DMSO/CDCl₃): δ=14.54; 29.40; 30.52; 50.78; 51.79; 54.61; 65.35; 127.47; 127.61; 128.20; 134.53; 136.92; 155.57; 171.39; 171.94

6i) Z-L-Trp-D-Met-OMe (Z-LD-Ij-OMe)

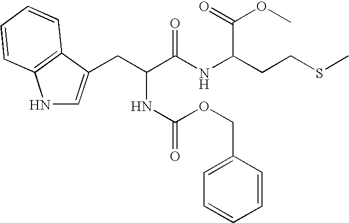

Empirical formula: C₂₅H₂₉N₃O₅S (483.58 g/mol), yield: 5.50 g (57%), purity: 99% (HPLC), slightly yellowish solid

¹H-NMR of Z-L-Trp-D-Met-OMe (Z-LD-Ij-OMe) (500 MHz, CDCl₃): δ=1.68-1.92 (m, 2H, SCH₂CH₂); 1.97 (s, 3H, SCH₃); 2.08-2.14 (m, 2H, SCH₂); 3.14-3.34 (m, 2H, CH₂-indolyl); 3.64 (s, 3H, OCH₃); 4.50-4.62 (m, 2H, 2×CH); 5.10 (s, 2H, OCH₂); 5.44 (bs, 1H, NH); 6.32 (bs, 1H, NH); 7.00-7.38, 10H; aromat.); 8.17 (bs, 1H, NH)

¹³C-NMR of Z-L-Trp-D-Met-OMe (Z-LD-Ij-OMe) (125 MHz, CDCl₃): δ=15.31; 29.48; 31.26; 33.97; 51.48; 52.45; 55.65; 67.10; 1101.37; 111.34; 118.77; 119.94; 122.44; 123.14; 127.32; 128.09; 128.22; 128.56; 136.20; 136.28; 155.99; 171.15; 171.80

6j) BOC-L-Trp-D-Met-OMe (BOC-LD-Ij-OMe)

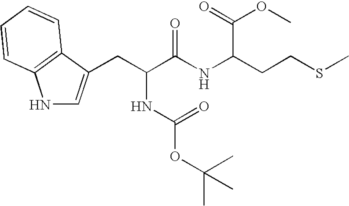

Empirical formula: C₂₂H₃₁N₃O₅S (449.56 g/mol), yield: 5.91 g (66%), purity: 99% (HPLC), white solid

¹H-NMR of BOC-L-Trp-D-Met-OMe (BOC-LD-Ij-OMe) (500 MHz, CDCl₃): δ=1.42 (s, 8H, C(CH₃)₃); 1.70-1.98 (m, 2H, SCH₂CH₂); 1.99 (s, 3H, SCH₃); 2.10-2.20 (m, 2H, SCH₂); 3.14-3.34 (m, 2H, CH₂-indolyl); 3.66 (s, 3H, OCH₃); 4.44-4.52 (m, 1H, CH); 4.56-4.62 (m, 1H, CH); 5.12 (bs, 1H, NH); 6.39 (d, ³J=8.0 Hz, 1H, NH); 7.04-7.38 (m, 5H, indolyl-CH); 8.17 (d, ³J=7.9 Hz, 1H, NH)

¹³C-NMR of BOC-L-Trp-D-Met-OMe (BOC-LD-Ij-OMe) (125 MHz, CDCl₃): δ=15.28; 28.27; 29.43; 31.36; 33.93; 52.38; 55.25; 80.19; 110.54; 111.25; 118.78; 119.80; 122.31; 123.06; 127.40; 136.25; 155.40; 171.53; 171.85

### Example 7

### General Specification for Synthesis of Compounds of the Group PG-L-EAA-D-Met (PG-LD-I) and PG-D-Met-L-EAA (PG-DL-II) (Methyl Ester Cleavage)

10.0 mmol of PG-L-EAA-D-Met-OMe (PG-LD-1-OMe) or PG-D-Met-L-EAA-OMe (PG-DL-II-OMe) was suspended in 15 ml of water and 200 ml methanol and 1.2 eq. (12.0 mmol) of NaOH (12.0 ml 1N NaOH) was added. After stirring for 2 hours, the homogeneous reaction solution was acidified with dilute hydrochloric acid and the methanol was partially distilled in the rotary evaporator. The white solid that crystallized out was filtered off, washed with 20 ml of water and recrystallized.

PG: protecting group (Z or BOC protecting group)

### Example 8

### General Specification for Synthesis of Compounds of the Group L-EAA-D-Met (LD-I) and D-Met-L-EAA (DL-II) (N-Terminal Z Protecting Group Cleavage)

5.0 mmol of Z-L-EAA-D-Met (Z-LD-I) or Z-D-Met-L-EAA (Z-LD-II) was dissolved in 50 ml of glacial acetic acid, and 18.5 ml (15.6 g; 250 mmol; 50 eq.) of dimethylsulphide and 5.0 g (3.6 ml) of 33% HBr in acetic acid (1.65 g; 4.0 eq.) were added. On completion of reaction, the reaction solution was concentrated in the rotary evaporator. The residue was dissolved in approx. 50 ml methanol and 3.5 g (50 mmol; 10 eq.) of sodium methane thiolate was added. After stirring for 20 minutes, the solution was neutralized at room temperature with concentrated hydrochloric acid and the solution was concentrated in the rotary evaporator. The residue was taken up in 40 ml of water and extracted three times with 40 ml diethyl ether each time. The aqueous phase was concentrated in the rotary evaporator: a voluminous white solid was precipitated. The dipeptide was removed with suction, washed with a little water and dried under vacuum.

### Example 9

### General Specification for Synthesis of Compounds of the Group L-EAA-D-Met (LD-I) and D-Met-L-EAA (DL-II) (N-Terminal BOC Protecting Group Cleavage)

5.0 mmol BOC-L-EAA-D-Met (BOC-LD-I) or BOC-D-Met-L-EAA (BOC-DL-II) was dissolved in 50 ml glacial acetic acid and 5.0 g (3.6 ml) of 33% HBr in acetic acid (1.65 g (4.0 eq.)) was added On completion of reaction, the reaction solution was concentrated in a rotary evaporator. The residue was taken up in 40 ml of water and extracted three times with 40 ml diethyl ether each time. The aqueous phase was slowly neutralized with 20% NaOH solution, while cooling continuously on an ice bath. The solution was washed three times with 40 ml diethyl ether each time and the aqueous phase was concentrated in the rotary evaporator, with precipitation of a voluminous white solid. The dipeptide was drawn off by suction, washed with a little water and dried under vacuum.

9a) D-Met-L-Leu (DL-IIb)

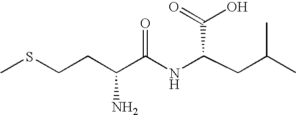

Yield: 860 mg (66%), purity: 98% (HPLC), voluminous white solid

¹H-NMR of H-D-Met-L-Leu (DL-IIb) (500 MHz, d₆-DMSO+HCl): δ=0.85 (d, ³J=6.3 Hz, 3H, CH₃); 0.90 (d, ³J=6.3 Hz, 3H, CH₃); 1.50-1.70 (m, 3H, SCH₂CH₂, CH(CH₃)₂); 2.00-2.10 (m, 5H, SCH₃, CH₂CH); 2.45-2.55 (m, 2H, SCH₂); 3.88-3.94 (m, 1H, CH); 4.22-4.30 (m, 1H, CH); 8.40-8.60 (m, 3H, NH₃⁺); 8.95 (d, ³J=8.3 Hz, 1H, NH)

¹³C-NMR of D-Met-L-Leu (DL-IIb) (500 MHz, d₆-DMSO+HCl): δ=14.56; 21.16; 22.95; 24.50; 28.21; 31.22; 50.66; 51.77; 168.16; 173.50

HRMS (pESI):

Calculated: 263.14294 C₁₁H₂₃N₂O₃S (MH⁺).

Found: 263.14224.

9b) D-Met-L-Ile (DL-IIc)

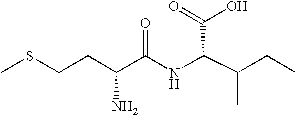

Yield: 900 mg (69%), purity: 99% (HPLC), voluminous white solid

¹H-NMR of D-Met-L-Ile (DL-IIc) (500 MHz, d₆-DMSO+HCl): δ=0.82-0.90 (m, 6H, 2×CH₃); 1.16-1.44 (m, 2H, SCH₂CH₃); 1.80-1.90 (m, 1H, CH); 2.00-2.10 (m, 2H, CH₂); 2.05 (s, 3H, SCH₃); 2.46-2.54 (m, 2H, SCH₂); 3.96-4.02 (m, 1H, CH); 4.24-4.30 (m, 1H, CH); 8.36-8.44 (m, 3H, NH₃⁺); 8.79 (d, ³J=8.5 Hz, 1H, NH)

¹³C-NMR of D-Met-L-Ile (DL-IIc) (500 MHz, d₆-DMSO+HCl): δ=11.44; 14.86; 15.96; 24.95; 28.58; 31.71; 36.75; 52.00; 56.82; 168.64; 172.74

HRMS (pESI):

Calculated: 263.14294 C₁₁H₂₃N₂O₃S (MH⁺).

Found: 263.14215.

9c) D-Met-L-Thr (DL-IId)

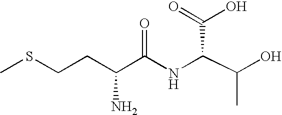

Yield: 640 mg (51%), purity: 98% (HPLC), voluminous white solid

¹H-NMR of D-Met-L-Thr (DL-IId) (500 MHz, d₆-DMSO+HCl): δ=1.10 (d, ³J=6.2 Hz, 3H, CHCH₃); 2.06 (s, 3H, SCH₃); 2.06-2.14 (m, 2H, SCH₂CH₂); 2.48-2.60 (m, 2H, SCH₂); 4.00-4.28 (m, 4H, 2×CH, CHOH); 8.40-8.46 (m, 3H, NH₃⁺); 8.77 (d, ³J=8.6 Hz, 1H, NH)

¹³C-NMR of D-Met-L-Thr (DL-IId) (500 MHz, d₆-DMSO+HCl): δ=15.14; 20.94; 28.74; 31.94; 52.44; 58.81; 66.97; 169.22; 172.20

HRMS (pESI):

Calculated: 251.10655 C₉H₁₉N₂O₄S (MH⁺).

Found: 251.10583.

9d) D-Met-L-Lys×2HCl (DL-Ile-2HCl)

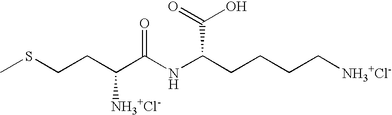

Yield: 613 mg (49%), purity: 97% (HPLC), yellowish solid

¹H-NMR of D-Met-L-Lys×2HCl (DL-Ile-2HCl) (500 MHz, DMSO): δ=1.32-1.42 (m, 2H, CH₂(Lys); 1.52-1.62 (m, 2H, CH₂(Lys); 1.64-1.80 (m, 2H, CH₂(Lys); 2.00-2.10 (m, 5H, SCH₂CH₂, SCH₃); 2.46-2.56 (m, 2H, SCH₂); 2.70-2.82 (m, 2H, NCH₂); 3.92-4.00 (m, 1H, CH); 4.16-4.24 (m, 1H, CH); 7.9 (bs, 3H, NH₃⁺); 8.3 (bs, 3H, NH₃⁺); 8.92 (d, ³J=7.7 Hz, 1H, NH)

HRMS (pESI):

Calculated: 278.15384 C₁₁H₂₄O₃S (MH⁺).

Found: 278.15288.

9e) D-Met-L-Phe (DL-IIg)

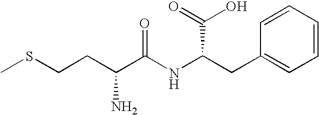

Yield: 930 mg (63%), purity: 98% (HPLC), voluminous white solid

¹H-NMR of D-Met-L-Phe (DL-IIg) (500 MHz, d₆-DMSO+HCl): δ=1.64-1.82 (m, 2H, SCH₂CH₂); 1.95 (s, 3H, SCH₃); 2.10-2.26 (m, 2H, SCH₂); 2.80-3.20 (m, 2H, CH₂Ph); 3.70 (t, ³J=6.1 Hz, 1H, CHCH₂Ph); 4.42-4.50 (m, 1H, CHCH₂CH₂S); 7.16-7.28 (m, 5H, Ph); 8.50-8.60 (bs, 1H, NH)

¹³C-NMR of D-Met-L-Phe (DL-IIg) (500 MHz, d₆-DMSO+HCl): δ=14.28; 28.08; 31.63; 37.03; 51.84; 53.78; 126.28; 127.97; 129.08; 137.69; 168.90; 172.65

HRMS (pESI):

Calculated: 297.12729 C₁₄H₂₁N₂O₃S (MH⁺).

Found: 297.12643.

9f) D-Met-L-Trp (DL-IIj)

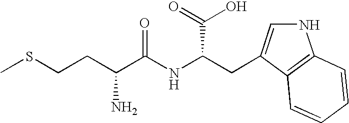

Yield: 1.38 g (82%), purity: 98% (HPLC), voluminous white solid

¹H-NMR of D-Met-L-Trp (DL-IIj) (500 MHz, d₆-DMSO+HCl): δ=1.50-1.80 (m, 2H, SCH₂CH₂); 1.93 (s, 3H, SCH₃); 2.30-2.40 (m, 2H, SCH₂); 3.02-3.22 (m, 2H, CH₂); 3.34-3.40 (m, 1H, SCH₂CH₂CH); 4.38-4.40 (m, 1H, CH); 6.90-7.60 (m, 5H, indolyl); 8.05-8.15 (bs, 1H, CONH); 10.80 (bs, 1H, NH)

¹³C-NMR of D-Met-L-Trp (DL-IIj) (500 MHz, d₆-DMSO+HCl): δ=14.37; 27.38; 29.12; 33.28; 53.00; 53.49; 110.26; 111.17; 118.07; 118.26; 120.64; 123.36; 127.52; 135.98; 171.87; 173.53

HRMS (pESI):

Calculated: 336.13819 C₁₆H₂₂N₃O₃S (MH⁺).

Found: 336.13718.

9g) L-Leu-D-Met (LD-Ib)

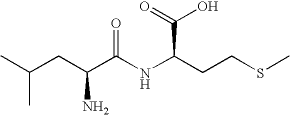

Yield: 710 mg (54%), purity: 99% (HPLC), voluminous white solid

¹H-NMR of H-L-Leu-D-Met (LD-Ib) (500 MHz, d₆-DMSO+HCl): δ=0.91 (t, ³J=5.4 Hz, 6H, 2×CH₃); 1.62 (t, ³J=6.8 Hz, 2H, CH₂CH(CH₃)₂); 1.60-1.75 (m, 1H, CH(CH₃)₂); 1.88-2.04 (m, 2H, SCH₂CH₂); 2.04 (s, 3H, SCH₃); 2.40-2.54 (m, 2H, SCH₂); 3.78-3.86 (m, 1H, CH); 4.32-4.40 (m, 1H, CH); 8.36 (d, ³J=4.0 Hz, 3H, NH₃⁺); 9.03 (d, ³J=7.8 Hz, 1H, NH)

¹³C-NMR of H-L-Leu-D-Met (LD-Ib) (500 MHz, d₆-DMSO+HCl): δ=14.56; 22.78; 23.33; 23.93; 29.89; 30.58; 41.03; 51.40; 51.56; 169.41; 173.03

HRMS (pESI):

Calculated: 263.14294 C₁₁H₂₃N₂O₃S (MH⁺).

Found: 263.14218.

9h) L-Ile-D-Met (LD-Ic)

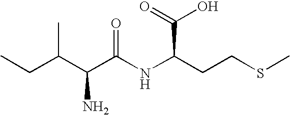

Yield: 790 mg (59%), purity: 97% (HPLC), voluminous white solid

¹H-NMR of L-Ile-D-Met (LD-Ic) (500 MHz, d₆-DMSO): δ=0.82 (t, ³J=7.4 Hz, 3H, CH₃CH₂); 0.86 (2, ³J=6.6 Hz, 3H, CH₃CH); 1.02-1.12 (m, 1H, CH₃CH/H″); 1.36-1.46 (m, 1H, CH₃CH/H″); 1.64-1.72 (m, 1H, CH₃CH); 1.80-1.98 (m, 2H, SCH₂CH₂); 2.00 (s, 3H, SCH₃); 2.36-2.44 (m, 2H, SCH₂); 3.27 (d, ³J=5.1 Hz, 1H, CH); 3.99 (t, ³J=5.3 Hz; 1H, CH); 7.92 (bs, 1H, NH)

¹³C-NMR of L-Ile-D-Met (LD-Ic) (500 MHz, d₆-DMSO): δ=11.57; 14.54; 15.60; 23.58; 29.69; 32.42; 37.90; 53.06; 58.79; 172.09; 173.37

HRMS (pESI):

Calculated: 263.14294 C₁₁H₂₃N₂O₃S (MH⁺).

Found: 263.14224.

9i) L-Thr-D-Met (LD-Id)

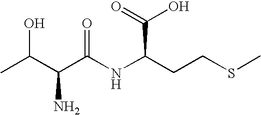

Yield: 690 mg (55%), purity: 99% (HPLC), voluminous white solid

¹H-NMR of L-Thr-D-Met (LD-Id) (500 MHz, d₆-DMSO+CDCl₃): δ=1.08 (d, ³J=6.6 Hz, 3H, CH₃); 1.82-2.08 (m, 2H, SCH₂CH₂), 2.02 (s, 3H, SCH₃); 2.38-2.50 (m, 2H, SCH₂); 3.06 (d, ³J=4.2 Hz, 1H, CH); 3.88-3.94 (m, 1H, CH); 3.98-4.04 (m, 1H, CH); 7.91 (d, ³J=7.3 Hz, 1H, NH)

¹³C-NMR of L-Thr-D-Met (LD-Id) (500 MHz, d₆-DMSO+CDCl₃): δ=14.75; 19.70; 30.07; 32.45; 53.71; 60.22; 67.45; 172.58; 174.24

HRMS (pESI):

Calculated: 251.10655 C₉H₁₉N₂O₄S (MH⁺).

Found: 251.10586.

9j) L-Lys-D-Met×2HCl (LD-Ie-2HCl)

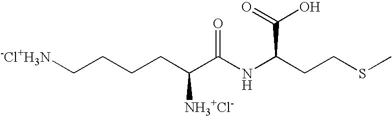

Yield: 676 mg (54%), purity: 96% (HPLC), colourless crystals

¹H-NMR of L-Lys-D-Met×2HCl (LD-Ie-2HCl) (500 MHz, d₆-DMSO): δ=1.30-1.44 (m, 2H, CH₂(Lys)); 1.54-1.64 (m, 2H, CH₂(Lys)); 1.72-1.84 (m, 1H, CH₂(Lys)); 1.90-2.04 (m, 2H, SCH₂CH₂); 2.05 (s, 3H, SCH₃); 2.44-2.58 (m, 2H, SCH₂); 2.70-2.80 (m, 2H, NCH₂); 3.82-3.90 (m, 1H, CH); 4.34-4.42 (m, 1H, CH); 7.9 (bs, 3H, NH₃⁺); 8.3 (bs, 3H, NH₃⁺); 8.91 (d, ³J=7.9 Hz, 1H, NH)

HRMS (pESI):

Calculated: 278.15384 C₁₁H₂₄O₃S (MH⁺).

Found: 278.15290.

9k) L-Phe-D-Met (LD-Ig)

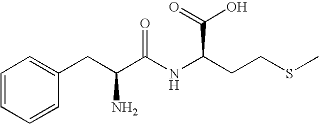

Yield: 880 mg (59%), purity: 98% (HPLC), voluminous white solid

¹H-NMR of L-Phe-D-Met (LD-Ig) (500 MHz, d₆-DMSO+D₂O): δ=1.60-2.02 (m, 4H, SCH₂CH₂); 2.05 (s, 3H, SCH₃); 3.08-3.32 (m, 2H, PhCH₂); 4.12-4.16 (m, 1H, CH); 4.20-4.26 (m, ¹H, CH); 7.30-7.50 (m, 5H, Ph)

¹³C-NMR of L-Phe-D-Met (LD-Ig) (500 MHz, d₆-DMSO+D₂O): δ=15.37; 30.72; 32.10; 38.09; 55.40; 55.96; 129.24; 130.50; 130.71; 136.55; 169.47; 178.42

HRMS (pESI):

Calculated: 297.12729 C₁₄H₂₁N₂O₃S (MH⁺).

Found: 297.12646.

9l) L-Trp-D-Met (LD-Ij)

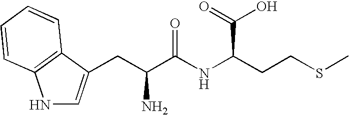

Yield: 1.40 g (83%), purity: 98% (HPLC), voluminous white solid

¹H-NMR of L-Trp-D-Met (LD-Ij) (500 MHz, d₆-DMSO): δ=1.68-1.88 (m, 2H, SCH₂CH₂); 1.94 (s, 3H, SCH₃); 2.24 (d, ³J=7.9 Hz, 2H, SCH₂); 2.80-2.88 (m, 1H, CH); 3.10-3.16 (m, 1H, CH); 3.70-3.76 (m, 1H, CH); 4.00-4.06 (m, 1H, CH); 6.90-7.60 (m, 5H, indolyl); 8.10 (bs, 1H, NH); 10.90 (bs, 1H, NH)

¹³C-NMR of L-Trp-D-Met (LD-Ij) (500 MHz, d₆-DMSO): δ=14.51; 29.56; 29.90; 32.09; 52.78; 54.59; 109.82; 111.26; 118.15; 118.30; 120.80; 123.82; 127.20; 136.16; 172.03; 173.02

HRMS (pESI):

Calculated: 336.13819 C₁₆H₂₂N₃O₃S (MH⁺).

Found: 336.13724.

### Example 10

### Chemical Synthesis of the diastereomeric mixture of Met-Ile (IIc) from 5-[2-(methylthio)ethyl]-2,4-imidazolidinedione (methionine hydantoin) (Vn) and L-isoleucine with KOH

11.8 g (0.09 mol) of L-isoleucine, 17.2 g (0.09 mol, purity: 91%) of 5-[2-(methylthio)ethyl]-2,4-imidazolidinedione (Vn) and 11.9 g (0.8 mol) of 85% KOH were dissolved in 150 ml of water and stirred in a 200 ml Roth steel autoclave with magnetic stirrer for 5 hours at 150° C., with increase in pressure to 8 bar. On completion of reaction the autoclave was cooled, the precipitated solid was filtered off and washed with a little water. A moderate CO₂ stream was passed through the filtrate. The solid that now precipitated was drawn off once again, washed with a little cold water and dried under oil-pump vacuum for several hours at 30° C.; final weight: 7.3 g (31% of theory) of white solid. ¹H-NMR coincided with the superimposed ¹H-NMR spectra of L-Met-L-Ile (LL-IIc) and D-Met-L-Ile (DL-IIc) (see Example 9b).

### Example 11

### Chemical Synthesis of the Diastereomeric Mixture of Met-Ile (IIc) from N-carbamoylmethionine (IIIn) and L-isoleucine with KOH

11.8 g (0.09 mol) of L-isoleucine, 17.5 g (0.09 mol, purity: 99%) of N-carbamoylmethionine (IIIn) and 11.9 g (0.18 mol) of 85% KOH were dissolved in 150 ml of water and stirred in a 200 ml Roth steel autoclave with magnetic stirrer for 5 hours at 150° C., with increase in pressure to 7 bar. On completion of reaction the autoclave was cooled, the precipitated solid was filtered off and washed with a little water. The filtrate was neutralized with 10% sulphuric acid and the solid that precipitated was drawn off by suction, washed with a little cold water and dried under oil-pump vacuum for several hours at 30° C.; final weight: 6.4 g (27% of theory) of white solid. ¹H-NMR coincided with the superimposed ¹H-NMR spectra of L-Met-L-Ile (LL-IIc) and D-Met-L-Ile (DL-IIc) (see Example 9b).

### Example 12

### Chemical synthesis of the diastereomeric mixture of Met-Ile (IIc) from 2-[(aminocarbonyl)amino]-4-(methylthio)butanoic acid amide (N-carbamoylmethioninamide) (IVn) and L-isoleucine with KOH

11.8 g (0.09 mol) of L-isoleucine, 17.4 g (90 mmol, purity: 98.5%) of 2-[(aminocarbonyl)amino]-4-(methylthio)butanoic acid amide (IVn) and 11.9 g (0.8 mol) of 85% KOH were dissolved in 150 ml of water and stirred in a 200 ml Roth steel autoclave with magnetic stirrer for 5 hours at 150° C., with increase in pressure to 7 bar. On completion of reaction the autoclave was cooled, the precipitated solid was filtered off and washed with a little water. The filtrate was neutralized with semi-concentrated hydrochloric acid and the solid that precipitated was drawn off by suction, washed with a little cold water and dried under oil-pump vacuum for several hours at 30° C.; final weight: 8.0 g (34% of theory) of white solid. ¹H-NMR coincided with the superimposed ¹H-NMR spectra of L-Met-L-Ile (LL-IIc) and D-Met-L-Ile (DL-IIc) (see Example 9b).

### Example 13

### Chemical synthesis of 3-[2-(methylthio)ethyl]-6-(1-(methyl)propyl)-2,5-piperazinedione (VIc) from 5-[2-(methylthio)ethyl]-2,4-imidazolidinedione (methionine hydantoin) (Vn) and L-isoleucine

11.8 g (0.09 mol) of L-isoleucine, 17.2 g (0.09 mol, purity: 91%) of 5-[2-(methylthio)ethyl]-2,4-imidazolidinedione (Vn) and 7.1 g (0.9 mol) of (NH₄)HCO₃ were dissolved in 150 ml of water and stirred in a 200 ml Roth steel autoclave with magnetic stirrer for 5 hours at 150° C., with increase in pressure. By releasing gas from time to time the pressure was kept constant at 8 bar. On completion of reaction the autoclave was cooled on an ice bath. The suspension obtained was then filtered, the solid filtered off was washed several times with water and dried under oil-pump vacuum for several hours at 30° C.; final weight: 9.9 g (45% of theory) of VIc as a white solid.

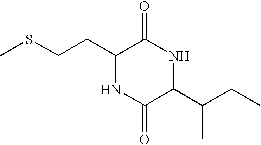

¹H-NMR of 3-[2-(methylthio)ethyl]-6-(1-(methyl)propyl)-2,5-piperazinedione (VIc) (500 MHz, d₆-DMSO): δ=0.85 (t, ³J=7.4 Hz, 3H, CH₂CH₃); 0.90 (d, ³J=7.4 Hz, 3H, CHCH₃); 1.10-1.50 (m, 2H, SCH₂CH₂); 1.80-1.90 (m, 1H, CH); 1.90-2.00 (m, 2H, CH₂); 2.04 (s, 3H, SCH₃); 2.42-2.58 (m, 2H, SCH₂); 3.64-3.68 (m, 1H, CH); 3.94-3.98 (m, 1H, CH); 8.08-8.16 (m, 2H, 2×NH)

¹³C-NMR of 3-[2-(methylthio)ethyl]-6-(1-(methyl)propyl)-2,5-piperazinedione (VIc) (500 MHz, d₆-DMSO+HCl): δ=12.02; 14.85; 15.27; 24.61; 28.74; 32.15; 39.90; 52.92; 59.34; 167.90; 168.10

### Example 14

### Chemical synthesis of 3-[2-(methylthio)ethyl]-6-(1-methyl)propyl)-2,5-piperazinedione (VIc) from N-carbamoylmethionine (IIIn) and L-isoleucine

11.8 g (0.09 mol) of L-isoleucine, 17.5 g (0.09 mol, purity: 99%) of N-carbamoylmethionine (IIIn) and 7.1 g (0.9 mol) of (NH₄)HCO₃ were dissolved in 150 ml of water and stirred in a 200 ml Roth steel autoclave with magnetic stirrer for 5 hours at 150° C., with increase in pressure. By releasing gas from time to time the pressure was kept constant at 8 bar. On completion of reaction the autoclave was cooled on an ice bath. The suspension obtained was then filtered, the solid filtered off was washed several times with water and dried under oil-pump vacuum for several hours at 30° C.; final weight: 9.1 g (41.3% of theory) of compound VIc as a white solid. NMR coincided with the NMR from Example 13.

### Example 15

### Chemical synthesis of 3-[2-(methylthio)ethyl]-6-(1-methyl)propyl)-2,5-piperazinedione (VIc) from 2-[(aminocarbonyl)amino]-4-(methylthio)butanoic acid amide (N-carbamoylmethioninamide) (IVn) and L-isoleucine

11.8 g (0.09 mol) of L-isoleucine, 17.4 g (90 mmol, purity: 98.5%) of 2-[(aminocarbonyl)amino]-4-(methylthio)butanoic acid amide (IVn) and 7.1 g (0.9 mol) of (NH₄)HCO₃ were dissolved in 150 ml of water and stirred in a 200 ml Roth steel autoclave with magnetic stirrer for 5 hours at 150° C., with increase in pressure. By releasing gas from time to time the pressure was kept constant at 8 bar. On completion of reaction the autoclave was cooled on an ice bath. The suspension obtained was then filtered, the solid filtered off was washed several times with water and dried under oil-pump vacuum for several hours at 30° C.; final weight: 10.3 g (47% of theory) of white solid IVc. NMR coincided with the NMR from Example 13.

### Example 16

### Synthesis of the diastereomeric mixture of Ile-Met (Ic) Met-Ile (IIc) from 3-[2-(methylthio)ethyl]-6-(1-methyl)propyl)-2,5-piperazinedione (VIc) with concentrated hydrochloric acid

24.4 g (100 mmol) of 3-[2-(methylthio)ethyl]-6-(1-methyl)propyl)-2,5-piperazinedione (VIc) was suspended in 66 g water. While stirring, 11 g conc. hydrochloric acid was slowly added dropwise and then heated carefully to reflux, stirring very vigorously. The reaction mixture was then heated under reflux for 8 hours, so that all of the solid went into solution. During subsequent cooling, a small amount of unreacted diketopiperazine was precipitated, and was filtered off. The filtrate was then adjusted to pH 5-6 with 32% ammonia water in a beaker on an ice bath. A mixture of DL-Met-DL-Ile (diastereomeric mixture of IIc) and DL-Ile-DL-Met (diastereomeric mixture of Ic) was precipitated as a voluminous white solid. The solid was dried in a drying cabinet at 40° C. under water-jet-pump vacuum; yield: 21.5 g (82.0%).

### Example 17

### Synthesis of the diastereomeric mixture of Ile-Met (Ic) and Met-Ile (IIc) from 3-[2-(methylthio)ethyl]-6-(1-methyl)propyl)-2,5-piperazinedione (VIc) in alkaline conditions with ammonia

19.6 g (0.8 mol) of 3-[2-(methylthio)ethyl]-6-(1-methyl)propyl)-2,5-piperazinedione (VIc), 22.4 ml of 25% ammonia solution and 160 ml of water were heated in an autoclave at 150° C. for 2 hours. After cooling, the unreacted diketopiperazine was drawn with suction. This could be used again in a subsequent preparation. The filtrate was concentrated in a rotary evaporator at a water temperature of 80° C. until the first crystals were precipitated. After cooling and leaving to stand overnight, after filtration and drying, a mixture of DL-Met-DL-Ile (diastereomeric mixture of IIc) and DL-Ile-DL-Met (diastereomeric mixture of Ic) was isolated as a voluminous white solid; yield: 12.2 g (58%).

### Example 18

### In Vitro Digestion Tests on L-EAA-L-Met (LL-I) or L-Met-L-EAA (LL-II) with Digestive Enzymes from Omnivorous Carp

a) Isolation of the Digestive Enzymes from Mirror Carp (Cyprinus carpio morpha noblis)

The digestive enzymes were isolated according to the method of EID and MATTY (Aquaculture 1989, 79, 111-119). For this, the intestines were removed from five one-year-old mirror carp (Cyprinus carpio morpha noblis), rinsed with water, cut open lengthwise and in each case the intestinal mucosa was scraped off. This was comminuted in a mixer together with crushed ice. The resulting suspension was treated with an ultrasound rod, to disrupt any cells that were still intact. To separate the cell constituents and fat, the suspension was centrifuged for 30 minutes at 4° C., the homogenate was decanted off and sterilized with a trace of thiomersal. From 5 mirror carp, 296.3 ml of enzyme solution of the intestinal mucosa was obtained. The solution was stored in the dark at 4° C.

b) Procedure for the In Vitro Digestion Studies

L-Met-L-EAA (LL-II) or L-EAA-L-Met (LL-I) was taken up in TRIS/HCl buffer solution and the enzyme solution was added. As comparison and to assess the rate of purely chemical cleavage, in each case a blank was prepared without enzyme solution (see Table 3). A sample was taken from time to time and its composition was detected and quantified by means of a calibrated HPLC. The conversion was determined as the quotient of the content of methionine and the content of L-Met-L-EAA (LL-II) or L-EAA-L-Met (LL-I) (see FIGS. 1 and 2).

TABLE 3

| | | Sample | | Blank | |
|---|---|---|---|---|---|
| Charge | Substrate | 0.15 | mmol | 0.15 | mmol |
| | (LL-I or LL-II) | | | | |
| | TRIS/HCl buffer | 7.5 | ml | 8.1 | ml |
| | solution, pH 9.5 | | | | |
| Start of | Enzyme solution | 589 | μl | — | |
| reaction | ( [symbol] 1.5% carp solution) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

### Example 19

### In Vitro Digestion Tests on L-EAA-D-Met (LD-I) or D-Met-L-EAA (DL-II) with Digestive Enzymes from Omnivorous Carp

a) Isolation of the Digestive Enzymes from Mirror Carp (Cyprinus carpio morpha noblis)

The digestive enzymes were isolated according to the method of EID and MATTY (Aquaculture 1989, 79, 111-119). For this, the intestines were removed from five one-year-old mirror carp (Cyprinus carpio morpha noblis) and processed as described in Example 18.

b) Procedure for the In Vitro Digestion Studies

D-Met-L-EAA (DL-II) or L-EAA-D-Met (LD-I) was taken up in TRIS/HCl buffer solution and the enzyme solution was added. As comparison and to assess the rate of purely chemical cleavage, a blank without enzyme solution was prepared in each case (see Table 4). A sample was taken from time to time and its composition was detected and quantified by means of a calibrated HPLC. The conversion was determined as the quotient of the area of methionine and the area of D-Met-L-EAA (DL-II) or L-EAA-D-Met (LD-I) (see FIG. 7).

TABLE 4

| | | Sample | | Blank | |
|---|---|---|---|---|---|
| Charge | Substrate | 0.15 | mmol | 0.15 | mmol |
| | (LD-I or DL-II) | | | | |
| | TRIS/HCl buffer | 7.5 | ml | 13.4 | ml |
| | solution, pH 9.5 | | | | |
| Start of | Enzyme solution | 5.89 | ml | — | |
| reaction | ( [symbol] 15% carp solution) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

### Example 20

### In Vitro Digestion Tests on L-EAA-L-Met (LL-I) or L-Met-L-EAA (LL-II) with Digestive Enzymes from Carnivorous Trout

a) Isolation of the Digestive Enzymes from Rainbow Trout (Oncorhynchus mykiss)

The digestive enzymes were isolated according to the method of EID and MATTY (Aquaculture 1989, 79, 111-119). For this, the intestines were removed from six one-year-old rainbow trout (Oncorhynchus mykiss) and processed as described in Example 18.

b) Procedure for the In Vitro Digestion Studies

The in vitro investigations were carried out similarly to Example 18 (see Table 5, FIGS. 3 and 4).

TABLE 5

| | | Sample | | Blank | |
|---|---|---|---|---|---|
| Charge | Substrate | 0.15 | mmol | 0.15 | mmol |
| | (LL-I or LL-II) | | | | |
| | TRIS/HCl buffer | 7.5 | ml | 7.9 | ml |
| | solution, pH 9.5 | | | | |
| Start of | Enzyme solution | 424 | μl | — | |
| reaction | ( [symbol] 1.0% trout solution) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

### Example 21

### In Vitro Digestion Tests on L-EAA-D-Met (LD-I) or D-Met-L-EAA (DL-II) with Digestive Enzymes from Carnivorous Trout

a) Isolation of the Digestive Enzymes from Rainbow Trout (Oncorhynchus mykiss)

The digestive enzymes were isolated according to the method of EID and MATTY (Aquaculture 1989, 79, 111-119). For this, the intestines were removed from six one-year-old rainbow trout (Oncorhynchus mykiss) and processed as described in Example 18.

b) Procedure for the In Vitro Digestion Studies

The in vitro investigations were carried out similarly to Example 19 (see Table 6, FIG. 11).

TABLE 6

| | | Sample | | Blank | |
|---|---|---|---|---|---|
| Charge | Substrate | 0.143 | mmol | 0.143 | mmol |
| | (LD-I or DL-II) | (40.1 | mg) | (40.1 | mg) |
| | TRIS/HCl buffer | 5.7 | ml | 9.9 | ml |
| | solution, pH 9.5 | | | | |
| Start of | Enzyme solution | 4.2 | ml | — | |
| reaction | ( [symbol] 10% trout solution) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

### Example 22

### In Vitro Digestion Tests on L-EAA-L-Met (LL-I) or L-Met-L-EAA (LL-II) with Digestive Enzymes from Omnivorous Shrimps

a) Isolation of the Digestive Enzymes from Whiteleg Shrimps (Litopenaeus vannamei)

The digestive enzymes were isolated according to the method of Ezquerra and Garcia-Carreno (J. Food Biochem. 1999, 23, 59-74). For this, the hepatopancreas was removed from five kilograms of whiteleg shrimps (Litopenaeus vannamei) and comminuted in a mixer together with crushed ice. Further processing was carried out similarly to Example 18.

b) Procedure for the In Vitro Digestion Studies

The in vitro investigations were carried out similarly to Example 18 (see Table 7, FIGS. 5 and 6).

TABLE 7

| | |
|---|---|
| Sample | Blank |

| Charge | Substrate | 0.15 | mmol | 0.15 | mmol |
|---|---|---|---|---|---|
| | (LL-I or LL-II) | | | | |
| | TRIS/HCl buffer | 7.5 | ml | 7.8 | ml |
| | solution, pH 9.5 | | | | |
| Start of | Enzyme solution | 258 | μl | — | |
| reaction | ( [symbol] 2 shrimps) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

### Example 23

### In Vitro Digestion Tests on L-EAA-D-Met (LD-I) or D-Met-L-EAA (DL-II) with Digestive Enzymes from Omnivorous Shrimps

a) Isolation of the Digestive Enzymes from Whiteleg Shrimps (Litopenaeus vannamei)

The digestive enzymes were isolated according to the method of Ezquerra and Garcia-Carreno (J. Food Biochem. 1999, 23, 59-74). For this, the hepatopancreas was removed from five kilograms of whiteleg shrimps (Litopenaeus vannamei) and comminuted in a mixer together with crushed ice. Further processing was carried out similarly to Example 18.

b) Procedure for the In Vitro Digestion Studies

The in vitro investigations were carried out similarly to Example 19 (see Table 8, FIG. 10).

TABLE 8

| | |
|---|---|
| Sample | Blank |

| Charge | Substrate | 0.143 | mmol | 0.143 | mmol |
|---|---|---|---|---|---|
| | (LD-I or DL-II) | (40.1 | mg) | (40.1 | mg) |
| | TRIS/HCl buffer | 5.7 | ml | 7.9 | ml |
| | solution, pH 9.5 | | | | |
| Start of | Enzyme solution | 2.2 | ml | — | |
| reaction | ( [symbol] 8 shrimps) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

### Example 24

### In Vitro Digestion Tests on L-EAA-L-Met (LL-I) or L-Met-L-EAA (LL-II) with Digestive Enzymes from Chicken

a) Isolation of the Digestive Enzymes from Chicken

The digestive enzymes were isolated according to the method of EID and MATTY (Aquaculture 1989, 79, 111-119). For this, the intestines were removed from a chicken, rinsed in water, cut open lengthwise and in each case the intestinal mucosa was scraped off. This was comminuted in a mixer together with crushed ice. The resulting suspension was treated with an ultrasound rod, to disrupt cells that were still intact. To separate cell constituents and fat, the suspension was centrifuged for 30 minutes at 4° C., the homogenate was decanted and sterilized with a trace of thiomersal. From one chicken, 118.9 ml of enzyme solution from the intestinal mucosa was obtained; the solution was stored in the dark at 4° C.

b) Procedure for the In Vitro Digestion Studies

L-Met-L-EAA (LL-II) or L-EAA-L-Met (LL-I) was taken up in TRIS/HCl buffer solution and the enzyme solution was added. As comparison and to assess the rate of purely chemical cleavage, a blank without enzyme solution was prepared in each case. A sample was taken from time to time and its composition was detected and quantified by means of a calibrated HPLC. The conversion was determined as the quotient of the content of methionine and of the content of L-Met-L-EAA (LL-II) or L-EAA-L-Met (LL-I) (see Table 9, FIG. 16).

TABLE 9

| | | Sample | | Blank | |
|---|---|---|---|---|---|
| Charge | Substrate | 0.15 | mmol | 0.15 | mmol |
| | (LL-I or LL-II) | | | | |
| | TRIS/HCl buffer | 11.3 | ml | 12.5 | ml |
| | solution, pH 9.5 | | | | |
| Start of | Enzyme solution | 1.19 | ml | — | |
| reaction | ( [symbol] 1.0% chicken solution) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

### Example 25

### In Vitro Digestion Tests on L-EAA-D-Met (LD-I) or D-Met-L-EAA (DL-II) with Digestive Enzymes from Chicken

a) Isolation of the Digestive Enzymes from Chicken

The digestive enzymes were isolated according to the method of EID and MATTY (Aquaculture 1989, 79, 111-119). For this, the intestines were removed from a chicken and processed as described in Example 24.

b) Procedure for the In Vitro Digestion Studies

D-Met-L-EAA (DL-II) or L-EAA-D-Met (LD-I) was taken up in TRIS/HCl buffer solution and the enzyme solution was added. As comparison and to assess the rate of purely chemical cleavage, a blank without enzyme solution was prepared in each case. A sample was taken from time to time and its composition was detected and quantified by means of a calibrated HPLC. The conversion was determined as the quotient of the area of methionine and the area of D-Met-L-EAA (DL-II) or L-EAA-D-Met (LD-I) (see Table 10, FIG. 17).

TABLE 10

| | | Sample | | Blank | |
|---|---|---|---|---|---|
| Charge | Substrate | 0.15 | mmol | 0.15 | mmol |
| | (LD-I or DL-II) | | | | |
| | TRIS/HCl buffer | 11.3 | ml | 12.5 | ml |
| | solution, pH 9.5 | | | | |
| Start of | Enzyme solution | 1.19 | ml | — | |
| reaction | ( [symbol] 1% chicken solution) | | | | |
| Reaction | | 37° | C. | 37° | C. |

| | |
|---|---|
| Stopping of | 0.2 ml of reaction solution was taken up in |
| the reaction | 9.8 ml of 10% H₃PO₄ solution. |

1. A composition, comprising dipeptides or salts thereof, wherein one amino acid residue of the dipeptide is a DL-methionyl residue and the other amino acid residue of the dipeptide is an amino acid in the L-configuration selected from the group consisting of lysine, threonine, tryptophan, histidine, valine, leucine, isoleucine, phenylalanine, arginine, cysteine and cystine.

2. The composition according to claim 1, comprising dipeptides of formula DL-methionyl-L-EAA, L-EAA-DL-methionine or both, wherein L-EAA is an amino acid in the L-configuration selected from the group consisting of lysine, threonine, tryptophan, histidine, valine, leucine, isoleucine, phenylalanine, arginine, cysteine and cystine.

3. A feed, comprising the composition according to claim 1; and a protein, carbohydrate or mixture thereof.

4. The feed according to claim 3, which comprises DL-methionyl-L-EAA, L-EAA-DL-methionine, D-methionyl-L-EAA, L-methionyl-L-EAA, L-EAA-D-methionine or L-EAA-L-methionine, alone, a mixture thereof or a mixture with D-methionyl-D-EAA, L-methionyl-D-EAA, D-EAA-D-methionine or D-EAA-L-methionine.

5. The feed according to claim 4, further comprising DL-methionine.

6. The feed according to claim 5, wherein DL-methionine is present in an amount from 0.01 to 90 wt. %.

7. The feed according to claim 5, wherein DL-methionine is present in an amount of from 0.1 to 50 wt. %.

8. The feed according to claim 5, wherein DL-methionine is present in an amount of from 1 to 30 wt. %.

9. The feed according to claim 5, which comprises L-EAA in an amount of from 0.01 to 90 wt. %.

10. The feed according to claim 9, wherein the L-EAA is present in an amount of from 0.1 to 50 wt. %.

11. The feed according to claim 9, wherein the L-EAA is present in an amount of from 1 to 30 wt. %.

12. A dipeptide or a salt thereof of formula DL-methionyl-DL-EAA or DL-EAA-DL-methionine, wherein EAA is an amino acid.

13. The dipeptide according to claim 12, wherein the amino acid is in the L-configuration and the amino acid is selected from the group consisting of lysine, threonine, tryptophan, histidine, valine, leucine, isoleucine, phenylalanine, arginine, cysteine and cystine.

14. A method of producing a dipeptide containing only one methionyl residue according to the formula DD/LL/DL/LD-I or DD/LL/DL/LD-II: 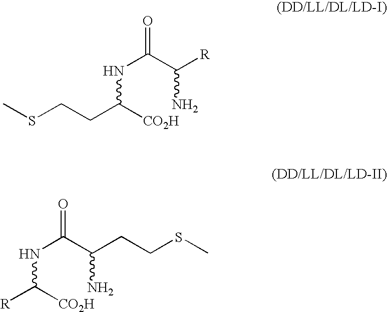
the method, comprising reacting an amino acid with a urea compound of formula III to V, 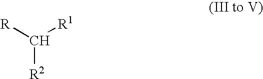
where R is defined as follows:

| | | |
|---|---|---|
| Ia to Va: | R = 1-methylethyl- | (valine) |
| Ib to Vb: | R = 2-methylpropyl- | (leucine) |
| Ic to Vc: | R = (1S)-1-methylpropyl- | (isoleucine) |
| Id to Vd: | R = (1R)-1-hydroxyethyl- | (threonine) |
| Ie to Ve: | R = 4-aminobutyl- | (lysine) |
| If to Vf: | R = 3-[(aminoiminomethyl)-amino]propyl- | (arginine) |
| Ig to Vg: | R = benzyl- | (phenylalanine) |
| Ih to Vh: | R = (1H-imidazol-4-yl)methyl- | (histidine) |
| Ij to Vj: | R = (1H-indol-3-yl)methyl- | (tryptophan) |
| Ik to Vk: | R = —CH₂—SH | (cysteine) |
| Im to Vm: | R = —CH₂—S—S—CH₂—CNH₂—COOH | (cystine) |
| IIIn to Vn: | R = —CH₂—CH₂—S—CH₃ | (methionine) |

with the residues R¹ and R² in the urea compound III, IV and V are as follows:
where
IIIa-n: R¹═COOH, R²═NHCONH₂
IVa-n: R¹═CONH₂, R²═NHCONH₂
Va-n: R¹-R²═—CONHCONH—
and
where R either denotes a methionyl residue and the added amino acid is selected from the group comprising lysine, threonine, tryptophan, histidine, valine, leucine, isoleucine, phenylalanine, arginine, cysteine and cystine
or
the added amino acid is methionine and R is an amino acid residue selected from the group consisting of lysine, threonine, tryptophan, histidine, valine, leucine, isoleucine, phenylalanine, arginine, cysteine and cystine.

15. The method according to claim 15, wherein the amino acid or an intermediate is methionine hydantoin or the hydantoin of an amino acid selected from the group consisting of lysine, threonine, tryptophan, histidine, valine, leucine, isoleucine, phenylalanine, arginine, cysteine, cystine.

16. The method according to claim 14, wherein a solution containing methionine hydantoin and water is reacted with the amino acid under basic conditions, or a solution containing the hydantoin of the amino acid selected from the group consisting of lysine, threonine, tryptophan, histidine, valine, leucine, isoleucine, phenylalanine, arginine, cysteine, cystine and water is reacted with methionine under basic conditions.

17. The method according to claim 14, wherein the urea compound is in a solution and the pH value of the solution is from 7 to 14, the reacting is carried out at a temperature of 30 to 200° C., the reaction is carried out at a pressure of 2 to 100 bar, or a combination thereof.

18. The method according to claim 15, wherein the methionine hydantoin or the hydantoin of the amino acid selected from the group consisting of lysine, threonine, tryptophan, histidine, valine, leucine, isoleucine, phenylalanine, arginine, cysteine, cystine and water is in a solution and wherein the solution was formed from one or more of the compounds III, IV and V.

19. The method according to claim 14, comprising:
a) reacting the urea compound according to formulae III, IV or V with the amino acid to form a diketopiperazine VI of formula, 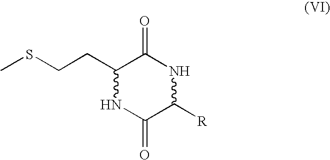
where R is defined as in claim 14; and
b) reacting the diketopiperazine VI with a mixture of dipeptides to form the formulae DD/LL/DL/LD-I and DD/LL/DL/LD-II: 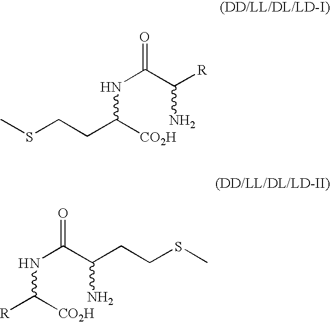
where R is defined as in claim 14.

20. The method according to claim 19, wherein the reaction of the urea compound with the amino acid to form the diketopiperazine is conducted at a temperature from 20° C. to 200° C., under pressure, preferably at a pressure from 2 to 90 bar, or a combination thereof.

21. The method according to claim 19, wherein the reaction of the urea compound with the amino acid to form the diketopiperazine is conducted in the presence of a base.

22. The method according to claim 21, wherein the base is selected from the group consisting of a nitrogen-containing base, NH₄HCO₃, (NH₄)₂CO₃, KHCO₃, K₂CO₃, NH₄OH/CO₂ mixture, a carbamate salt, an alkali base and an alkaline-earth base.

23. The method according to claim 19, wherein the reaction to form the diketopiperazine comprises reacting the urea compound of formula, 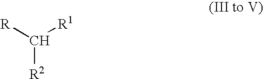
where R is a methionyl residue, with an amino acid selected from the group consisting of lysine, threonine, tryptophan, histidine, valine, leucine, isoleucine, phenylalanine, arginine, cysteine and cystine,
or
reacting the urea compound of formula, 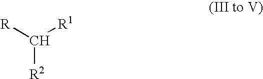
where R is an amino acid residue selected from the group consisting of lysine, threonine, tryptophan, histidine, valine, leucine, isoleucine, phenylalanine, arginine, cysteine and cystine, with methionine.

24. The method according to claim 19, wherein the reacting of the diketopiperazine to a mixture of dipeptides of formula I and II comprises acid hydrolysis.

25. The method according to claim 24, wherein the reacting is conducted in the presence of an acid selected from the group consisting of a mineral acid, HCl, H₂CO₃, CO₂/H₂O, H₂SO₄, a phosphoric acid, a carboxylic acid and a hydroxycarboxylic acid.

26. The method according to claim 19, wherein reacting of the diketopiperazine to a mixture of dipeptides of formula I and II comprises basic hydrolysis.

27. The method according to claim 26, wherein the reacting is conducted at a pH from 7 to 14.

28. The method according to claim 26, wherein the reacting is conducted in the presence of a base selected from the group consisting of a nitrogen-containing base, NH₄HCO₃, (NH₄)₂CO₃, NH₄OH/CO₂ mixture, a carbamate salt, KHCO₃, K₂CO₃, a carbonate, an alkali base and alkaline-earth base.

29. The method according to claim 14, wherein the urea compound of formula III to V is in the D-configuration, in the L-configuration or in a mixture of D- and L-configuration.

30. The method according to claim 29, wherein the urea compound is in a mixture of D- and L-configuration, if the urea compound is obtained from methionine (IIIn to Vn).

31. The method according to claim 29, wherein the urea compound of formula is in the L-configuration, if the urea compound III to V is obtained from an amino acid selected from the group consisting of lysine, threonine, tryptophan, histidine, valine, leucine, isoleucine, phenylalanine, arginine, cysteine, and cystine.

32. The method according to claim 26, further comprising crystallizing the basic reaction solution to isolate the diastereomeric mixture of the dipeptides of formula I and II.

33. The method according to claim 32, wherein crystallizing comprises adjusting the pH of the solution to from 2 to 10.

34. The method according to claim 32, wherein crystallizing comprises adjusting the pH of the solution to from 3 to 9.

35. The method according to claim 32, wherein crystallizing comprises adjusting the pH of the solution to a corresponding isoelectric point of the dieptide of formula I or II.

36. The method according to claim 33, wherein the adjusting the pH comprises adding an acid to the basic solution, wherein the acid is selected from the group consisting of a mineral acid, HCl, H₂CO₃, CO₂/H₂O, H₂SO₄, a phosphoric acid, a carboxylic acid and a hydroxycarboxylic acid.

37. The method according to claim 24, further comprising crystallizing the acidic reaction solution to isolate a diastereomeric mixture of the dipeptides of formula I and II.

38. The method according to claim 37, wherein crystallizing comprises adjusting the pH of the acidic solution a pH of from 2 to 10.

39. The method according to claim 37, wherein crystallizing comprises adjusting the pH of the acidic solution to a pH of from 3 to 9.

40. The method according to claim 37, wherein crystallizing comprises adjusting the acidic solution to a corresponding isoelectric point of the dipeptides of formula I or II.

41. The method according to claim 38, wherein adjusting the pH comprises adding a base to the acidic solution, wherein the base is selected from the group consisting of NH₄HCO₃, (NH₄)₂CO₃, a nitrogen-containing base, NH₄OH, a carbamate salt, KHCO₃, K₂CO₃, a carbonate, an alkali base and an alkaline-earth base.

42. A method of feeding an animal, the method comprising providing the compounds I and II obtained by the method according to claim 14.

43. The method according to claim 42, wherein the animal is a poultry, a pig, a ruminant animal, a fresh-water fish, a salt water fish, Crustacea or a pet.
